SEQUENCE LISTING (424 sequences)

SEQ ID NO: 1 - DNA, 637 bp, Homo sapiens
aaatgcttgggcatcagaagtgggagctgtgatcctagcttgggggcagcacagggtaggcggccttctctctgctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgc

SEQ ID NO: 2 - DNA, 192 bp, Homo sapiens
gcggccttctctctgctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaa

SEQ ID NO: 3 - DNA, 29 bp, Homo sapiens
tgcacacaagaacttttctcggggtgtaa

SEQ ID NO: 4 - DNA, 76 bp, Homo sapiens
aaggggcgcagcgccttcagcaaccggagaagcatagttgcacgcgacctggtgtgtgatctccgagtgggtgggg

SEQ ID NO: 5 - DNA, 22 bp, Homo sapiens
ttgcagaagagacccggaaagg

SEQ ID NO: 6 - DNA, 21 bp, Artificial Sequence
  Synthetic DNA/RNA construct
gcggccuucucucugcuuutt

SEQ ID NO: 7 - DNA, 21 bp, Artificial Sequence
  Synthetic DNA/RNA construct
cagaagagacccggaaaggtt

SEQ ID NO: 8 - DNA, 21 bp, Artificial Sequence
  Synthetic DNA/RNA construct
ccggagaagcauaguugcatt

SEQ ID NO: 9 - DNA, 21 bp, Artificial Sequence
  Synthetic DNA/RNA construct
cgcgaccuggugugugauctt

SEQ ID NO: 10 - DNA, 21 bp, Artificial Sequence
  Synthetic DNA/RNA construct
gagcaggcuugggaguccutt

SEQ ID NO: 11 - DNA, 21 bp, Artificial Sequence
  Synthetic DNA/RNA construct
uaguugcacgcgaccuggutt

SEQ ID NO: 12 - DNA, 21 bp, Artificial Sequence
  Synthetic DNA/RNA construct
gguccagaaucgcccagagtt

SEQ ID NO: 13 - DNA, 21 bp, Artificial Sequence
  Synthetic DNA/RNA construct
gcagaagagacccggaaagtt

SEQ ID NO: 14 - DNA, 21 bp, Artificial Sequence
  Synthetic DNA/RNA construct
ccgcggucgugcacaucugtt

SEQ ID NO: 15 - DNA, 21 bp, Artificial Sequence
  Synthetic DNA/RNA construct
cggguccagaaucgcccagtt

SEQ ID NO: 16 - DNA, 21 bp, Artificial Sequence
  Synthetic DNA/RNA construct
gccuucagcaaccggagaatt

SEQ ID NO: 17 - DNA, 21 bp, Artificial Sequence
  Synthetic DNA/RNA construct
acaccggagucgaauuacatt

SEQ ID NO: 18 - DNA, 21 bp, Artificial Sequence
  Synthetic DNA/RNA construct
aacuuuucucgggguguaatt

SEQ ID NO: 19 - DNA, 21 bp, Artificial Sequence
  Synthetic DNA/RNA construct
uugggaguccucugcacactt

SEQ ID NO: 20 - DNA, 21 bp, Artificial Sequence
  Synthetic DNA/RNA construct
uugggcaccagccgagagctt

SEQ ID NO: 21 - DNA, 21 bp, Artificial Sequence
  Synthetic DNA/RNA construct
ugcagaagagacccggaaatt

SEQ ID NO: 22 - DNA, 21 bp, Artificial Sequence
  Synthetic DNA/RNA construct
ccagaaucgcccagagccgtt

SEQ ID NO: 23 - DNA, 21 bp, Artificial Sequence
  Synthetic DNA/RNA construct
ggcgcagcgccuucagcaatt

SEQ ID NO: 24 - DNA, 21 bp, Artificial Sequence
  Synthetic DNA/RNA construct
cacgcgaccuggugugugatt

SEQ ID NO: 25 - DNA, 21 bp, Artificial Sequence
  Synthetic DNA/RNA construct
gcggguccagaaucgcccatt

SEQ ID NO: 26 - DNA, 21 bp, Artificial Sequence
  Synthetic DNA/RNA construct
uucucucugcuuugaguggtt

SEQ ID NO: 27 - DNA, 21 bp, Artificial Sequence
  Synthetic DNA/RNA construct
agccgagagccggcuccgctt

SEQ ID NO: 28 - DNA, 21 bp, Artificial Sequence
  Synthetic DNA/RNA construct
cacaucugacccgagucagtt

SEQ ID NO: 29 - DNA, 21 bp, Artificial Sequence
  Synthetic DNA/RNA construct
gccgagagccggcuccgcatt

SEQ ID NO: 30 - DNA, 21 bp, Artificial Sequence
  Synthetic DNA/RNA construct
gcuugggcaccagccgagatt

SEQ ID NO: 31 - DNA, 21 bp, Artificial Sequence
  Synthetic DNA/RNA construct
uugauuggcugcucgcacgtt

SEQ ID NO: 32 - DNA, 21 bp, Artificial Sequence
  Synthetic DNA/RNA construct
gccgggguggugacacacatt

SEQ ID NO: 33 - DNA, 21 bp, Artificial Sequence
  Synthetic DNA/RNA construct
ccuccauuggcugagaagatt

SEQ ID NO: 34 - DNA, 21 bp, Artificial Sequence
  Synthetic DNA/RNA construct
uucagcaaccggagaagcatt

SEQ ID NO: 35 - DNA, 21 bp, Artificial Sequence
  Synthetic DNA/RNA construct
ccuucagcaaccggagaagtt

SEQ ID NO: 36 - DNA, 21 bp, Artificial Sequence
  Synthetic DNA/RNA construct
gcaggcuugggaguccucutt

SEQ ID NO: 37 - DNA, 21 bp, Artificial Sequence
  Synthetic DNA/RNA construct
agcaggcuugggaguccuctt

SEQ ID NO: 38 - DNA, 21 bp, Artificial Sequence
  Synthetic DNA/RNA construct
agcuugggcaccagccgagtt

SEQ ID NO: 39 - DNA, 21 bp, Artificial Sequence
  Synthetic DNA/RNA construct
caggcuugggaguccucugtt

SEQ ID NO: 40 - DNA, 21 bp, Artificial Sequence
  Synthetic DNA/RNA construct
gcacaucugacccgagucatt

SEQ ID NO: 41 - DNA, 21 bp, Artificial Sequence
  Synthetic DNA/RNA construct
gcgccuucagcaaccggagtt

SEQ ID NO: 42 - DNA, 21 bp, Artificial Sequence
  Synthetic DNA/RNA construct
accuggugugugaucuccgtt

SEQ ID NO: 43 - DNA, 21 bp, Artificial Sequence
  Synthetic DNA/RNA construct
cgaauuacagcccugcaautt

SEQ ID NO: 44 - DNA, 21 bp, Artificial Sequence
  Synthetic DNA/RNA construct
ucgaauuacagcccugcaatt

SEQ ID NO: 45 - DNA, 21 bp, Artificial Sequence
  Synthetic DNA/RNA construct
cacacaagaacuuuucucgtt

SEQ ID NO: 46 - DNA, 21 bp, Artificial Sequence
  Synthetic DNA/RNA construct
ugggaguccucugcacacatt

SEQ ID NO: 47 - DNA, 21 bp, Artificial Sequence
  Synthetic DNA/RNA construct
gggagcugugauccuagcutt

SEQ ID NO: 48 - DNA, 21 bp, Artificial Sequence
  Synthetic DNA/RNA construct
cgccuucagcaaccggagatt

SEQ ID NO: 49 - DNA, 21 bp, Artificial Sequence
  Synthetic DNA/RNA construct
ggagcugugauccuagcuutt

SEQ ID NO: 50 - DNA, 21 bp, Artificial Sequence
  Synthetic DNA/RNA construct
gagucgaauuacagcccugtt

SEQ ID NO: 51 - DNA, 21 bp, Artificial Sequence
  Synthetic DNA/RNA construct
aaggggcgcagcgccuucatt

SEQ ID NO: 52 - DNA, 21 bp, Artificial Sequence
  Synthetic DNA/RNA construct
caccggagucgaauuacagtt

SEQ ID NO: 53 - DNA, 21 bp, Artificial Sequence
  Synthetic DNA/RNA construct
uccucugcacacaagaacutt

SEQ ID NO: 54 - DNA, 21 bp, Artificial Sequence
  Synthetic DNA/RNA construct
caccgcucccgcaccccagtt

SEQ ID NO: 55 - DNA, 21 bp, Artificial Sequence
  Synthetic DNA/RNA construct
guccucugcacacaagaactt

SEQ ID NO: 56 - DNA, 21 bp, Artificial Sequence
  Synthetic DNA/RNA construct
acaucugacccgagucagctt

SEQ ID NO: 57 - DNA, 21 bp, Artificial Sequence
  Synthetic DNA/RNA construct
cggccuucucucugcuuugtt

SEQ ID NO: 58 - DNA, 21 bp, Artificial Sequence
  Synthetic DNA/RNA construct
ggagacugaguggcgcgugtt

SEQ ID NO: 59 - DNA, 21 bp, Artificial Sequence
  Synthetic DNA/RNA construct
cuucagcaaccggagaagctt

SEQ ID NO: 60 - DNA, 21 bp, Artificial Sequence
  Synthetic DNA/RNA construct
aucgcccagagccgcccgctt

SEQ ID NO: 61 - DNA, 21 bp, Artificial Sequence
  Synthetic DNA/RNA construct
uucugggcgccuggcgggutt

SEQ ID NO: 62 - DNA, 21 bp, Artificial Sequence
  Synthetic DNA/RNA construct
uuggcugcucgcacgcgcctt

SEQ ID NO: 63 - DNA, 21 bp, Artificial Sequence
  Synthetic DNA/RNA construct
cuugggcaccagccgagagtt

SEQ ID NO: 64 - DNA, 21 bp, Artificial Sequence
  Synthetic DNA/RNA construct
ggcaucagaagugggagcutt

SEQ ID NO: 65 - DNA, 21 bp, Artificial Sequence
  Synthetic DNA/RNA construct
ggcggguccagaaucgccctt

SEQ ID NO: 66 - DNA, 21 bp, Artificial Sequence
  Synthetic DNA/RNA construct
caucagaagugggagcugutt

SEQ ID NO: 67 - DNA, 21 bp, Artificial Sequence
  Synthetic DNA/RNA construct
gcccgcggucgugcacauctt

SEQ ID NO: 68 - DNA, 21 bp, Artificial Sequence
  Synthetic DNA/RNA construct
uuacagcccugcaauuaactt

SEQ ID NO: 69 - DNA, 21 bp, Artificial Sequence
  Synthetic DNA/RNA construct
uugcagaagagacccggaatt

SEQ ID NO: 70 - DNA, 21 bp, Artificial Sequence
  Synthetic DNA/RNA construct
aggggcgcagcgccuucagtt

SEQ ID NO: 71 - DNA, 21 bp, Artificial Sequence
  Synthetic DNA/RNA construct
cucugcacacaagaacuuutt

SEQ ID NO: 72 - DNA, 21 bp, Artificial Sequence
  Synthetic DNA/RNA construct
ugaucuccgagugggugggtt

SEQ ID NO: 73 - DNA, 21 bp, Artificial Sequence
  Synthetic DNA/RNA construct
guugcagaagagacccggatt

SEQ ID NO: 74 - DNA, 21 bp, Artificial Sequence
  Synthetic DNA/RNA construct
cugcacacaagaacuuuuctt

SEQ ID NO: 75 - DNA, 21 bp, Artificial Sequence
  Synthetic DNA/RNA construct
cgagucagcuugggcaccatt

SEQ ID NO: 76 - DNA, 21 bp, Artificial Sequence
  Synthetic DNA/RNA construct
gucagcuugggcaccagcctt

SEQ ID NO: 77 - DNA, 21 bp, Artificial Sequence
  Synthetic DNA/RNA construct
ggagucgaauuacagcccutt

SEQ ID NO: 78 - DNA, 21 bp, Artificial Sequence
  Synthetic DNA/RNA construct
cgcggucgugcacaucugatt

SEQ ID NO: 79 - DNA, 21 bp, Artificial Sequence
  Synthetic DNA/RNA construct
uggugugugaucuccgagutt

SEQ ID NO: 80 - DNA, 21 bp, Artificial Sequence
  Synthetic DNA/RNA construct
ugcucgcacgcgccugccctt

SEQ ID NO: 81 - DNA, 21 bp, Artificial Sequence
  Synthetic DNA/RNA construct
ugcuugggcaucagaagugtt

SEQ ID NO: 82 - DNA, 21 bp, Artificial Sequence
  Synthetic DNA/RNA construct
aguggcuucugggcgccugtt

SEQ ID NO: 83 - DNA, 21 bp, Artificial Sequence
  Synthetic DNA/RNA construct
aguccucugcacacaagaatt

SEQ ID NO: 84 - DNA, 21 bp, Artificial Sequence
  Synthetic DNA/RNA construct
ugugugaucuccgagugggtt

SEQ ID NO: 85 - DNA, 21 bp, Artificial Sequence
  Synthetic DNA/RNA construct
ugagcaggcuugggagucctt

SEQ ID NO: 86 - DNA, 21 bp, Artificial Sequence
  Synthetic DNA/RNA construct
cgugcacaucugacccgagtt

SEQ ID NO: 87 - DNA, 21 bp, Artificial Sequence
  Synthetic DNA/RNA construct
ucgugcacaucugacccgatt

SEQ ID NO: 88 - DNA, 21 bp, Artificial Sequence
  Synthetic DNA/RNA construct
agcuucugauuggcucuuutt

SEQ ID NO: 89 - DNA, 21 bp, Artificial Sequence
  Synthetic DNA/RNA construct
gucgugcacaucugacccgtt

SEQ ID NO: 90 - DNA, 21 bp, Artificial Sequence
  Synthetic DNA/RNA construct
cacaggguaggcggccuuctt

SEQ ID NO: 91 - DNA, 21 bp, Artificial Sequence
  Synthetic DNA/RNA construct
cuggugugugaucuccgagtt

SEQ ID NO: 92 - DNA, 21 bp, Artificial Sequence
  Synthetic DNA/RNA construct
gcaccgcucccgcaccccatt

SEQ ID NO: 93 - DNA, 21 bp, Artificial Sequence
  Synthetic DNA/RNA construct
aagaacuuuucucggggugtt

SEQ ID NO: 94 - DNA, 21 bp, Artificial Sequence
  Synthetic DNA/RNA construct
cacaagaacuuuucucgggtt

SEQ ID NO: 95 - DNA, 21 bp, Artificial Sequence
  Synthetic DNA/RNA construct
gccuucucucugcuuugagtt

SEQ ID NO: 96 - DNA, 21 bp, Artificial Sequence
  Synthetic DNA/RNA construct
gagccggcuccgcaccgcutt

SEQ ID NO: 97 - DNA, 21 bp, Artificial Sequence
  Synthetic DNA/RNA construct
acgcgaccggcgcgaggagtt

SEQ ID NO: 98 - DNA, 21 bp, Artificial Sequence
  Synthetic DNA/RNA construct
aaaugcuugggcaucagaatt

SEQ ID NO: 99 - DNA, 21 bp, Artificial Sequence
  Synthetic DNA/RNA construct
gcuugggcaucagaaguggtt

SEQ ID NO: 100 - DNA, 21 bp, Artificial Sequence
  Synthetic DNA/RNA construct
agccgcccgcggucgugcatt

SEQ ID NO: 101 - DNA, 21 bp, Artificial Sequence
  Synthetic DNA/RNA construct
ggucgugcacaucugaccctt

SEQ ID NO: 102 - DNA, 21 bp, Artificial Sequence
  Synthetic DNA/RNA construct
ucagcaaccggagaagcautt

SEQ ID NO: 103 - DNA, 21 bp, Artificial Sequence
  Synthetic DNA/RNA construct
agaaucgcccagagccgcctt

SEQ ID NO: 104 - DNA, 21 bp, Artificial Sequence
  Synthetic DNA/RNA construct
gagcugugauccuagcuugtt

SEQ ID NO: 105 - DNA, 21 bp, Artificial Sequence
  Synthetic DNA/RNA construct
cggagaagcauaguugcactt

SEQ ID NO: 106 - DNA, 21 bp, Artificial Sequence
  Synthetic DNA/RNA construct
auaguugcacgcgaccuggtt

SEQ ID NO: 107 - DNA, 21 bp, Artificial Sequence
  Synthetic DNA/RNA construct
acuuuucucgggguguaaatt

SEQ ID NO: 108 - DNA, 21 bp, Artificial Sequence
  Synthetic DNA/RNA construct
cuucugggcgccuggcgggtt

SEQ ID NO: 109 - DNA, 21 bp, Artificial Sequence
  Synthetic DNA/RNA construct
cagccgccgggguggugactt

SEQ ID NO: 110 - DNA, 21 bp, Artificial Sequence
  Synthetic DNA/RNA construct
agcugugauccuagcuuggtt

SEQ ID NO: 111 - DNA, 21 bp, Artificial Sequence
  Synthetic DNA/RNA construct
gcgaccuggugugugaucutt

SEQ ID NO: 112 - DNA, 21 bp, Artificial Sequence
  Synthetic DNA/RNA construct
agcacaggguaggcggccutt

SEQ ID NO: 113 - DNA, 21 bp, Artificial Sequence
  Synthetic DNA/RNA construct
gcgccuggcggguccagaatt

SEQ ID NO: 114 - DNA, 21 bp, Artificial Sequence
  Synthetic DNA/RNA construct
gcaucagaagugggagcugtt

SEQ ID NO: 115 - DNA, 21 bp, Artificial Sequence
  Synthetic DNA/RNA construct
gaaucgcccagagccgccctt

SEQ ID NO: 116 - DNA, 21 bp, Artificial Sequence
  Synthetic DNA/RNA construct
gggcgcagcgccuucagcatt

SEQ ID NO: 117 - DNA, 21 bp, Artificial Sequence
  Synthetic DNA/RNA construct
uacagcccugcaauuaacatt

SEQ ID NO: 118 - DNA, 21 bp, Artificial Sequence
  Synthetic DNA/RNA construct
aacauaugaaucugacgaatt

SEQ ID NO: 119 - DNA, 21 bp, Artificial Sequence
  Synthetic DNA/RNA construct
accgcucccgcaccccagctt

SEQ ID NO: 120 - DNA, 21 bp, Artificial Sequence
  Synthetic DNA/RNA construct
agacugaguggcgcgugcctt

SEQ ID NO: 121 - DNA, 21 bp, Artificial Sequence
  Synthetic DNA/RNA construct
gcauaguugcacgcgaccutt

SEQ ID NO: 122 - DNA, 21 bp, Artificial Sequence
  Synthetic DNA/RNA construct
ugcacaucugacccgaguctt

SEQ ID NO: 123 - DNA, 21 bp, Artificial Sequence
  Synthetic DNA/RNA construct
aaucgcccagagccgcccgtt

SEQ ID NO: 124 - DNA, 21 bp, Artificial Sequence
  Synthetic DNA/RNA construct
cccgagucagcuugggcactt

SEQ ID NO: 125 - DNA, 21 bp, Artificial Sequence
  Synthetic DNA/RNA construct
ugggcaccagccgagagcctt

SEQ ID NO: 126 - DNA, 21 bp, Artificial Sequence
  Synthetic DNA/RNA construct
cuuuucucgggguguaaaatt

SEQ ID NO: 127 - DNA, 21 bp, Artificial Sequence
  Synthetic DNA/RNA construct
gcuuugaguggcuucugggtt

SEQ ID NO: 128 - DNA, 21 bp, Artificial Sequence
  Synthetic DNA/RNA construct
gucgaauuacagcccugcatt

SEQ ID NO: 129 - DNA, 21 bp, Artificial Sequence
  Synthetic DNA/RNA construct
gcggucgugcacaucugactt

SEQ ID NO: 130 - DNA, 21 bp, Artificial Sequence
  Synthetic DNA/RNA construct
aauuacagcccugcaauuatt

SEQ ID NO: 131 - DNA, 21 bp, Artificial Sequence
  Synthetic DNA/RNA construct
acguugcagaagagacccgtt

SEQ ID NO: 132 - DNA, 21 bp, Artificial Sequence
  Synthetic DNA/RNA construct
ggcgccuggcggguccagatt

SEQ ID NO: 133 - DNA, 21 bp, Artificial Sequence
  Synthetic DNA/RNA construct
gcacgcgaccuggugugugtt

SEQ ID NO: 134 - DNA, 21 bp, Artificial Sequence
  Synthetic DNA/RNA construct
ugcacgcgaccuggugugutt

SEQ ID NO: 135 - DNA, 21 bp, Artificial Sequence
  Synthetic DNA/RNA construct
ccgccgggguggugacacatt

SEQ ID NO: 136 - DNA, 21 bp, Artificial Sequence
  Synthetic DNA/RNA construct
ucuuugauuggcugcucgctt

SEQ ID NO: 137 - DNA, 21 bp, Artificial Sequence
  Synthetic DNA/RNA construct
acacaccggagucgaauuatt

SEQ ID NO: 138 - DNA, 21 bp, Artificial Sequence
  Synthetic DNA/RNA construct
aaugcuugggcaucagaagtt

SEQ ID NO: 139 - DNA, 21 bp, Artificial Sequence
  Synthetic DNA/RNA construct
aacucuuugauuggcugcutt

SEQ ID NO: 140 - DNA, 21 bp, Artificial Sequence
  Synthetic DNA/RNA construct
ggguccagaaucgcccagatt

SEQ ID NO: 141 - DNA, 21 bp, Artificial Sequence
  Synthetic DNA/RNA construct
cacaccggagucgaauuactt

SEQ ID NO: 142 - DNA, 21 bp, Artificial Sequence
  Synthetic DNA/RNA construct
gaguccucugcacacaagatt

SEQ ID NO: 143 - DNA, 21 bp, Artificial Sequence
  Synthetic DNA/RNA construct
aaagcagagagaaggccgctt

SEQ ID NO: 144 - DNA, 21 bp, Artificial Sequence
  Synthetic DNA/RNA construct
ccuuuccgggucucuucugtt

SEQ ID NO: 145 - DNA, 21 bp, Artificial Sequence
  Synthetic DNA/RNA construct
ugcaacuaugcuucuccggtt

SEQ ID NO: 146 - DNA, 21 bp, Artificial Sequence
  Synthetic DNA/RNA construct
gaucacacaccaggucgcgtt

SEQ ID NO: 147 - DNA, 21 bp, Artificial Sequence
  Synthetic DNA/RNA construct
aggacucccaagccugcuctt

SEQ ID NO: 148 - DNA, 21 bp, Artificial Sequence
  Synthetic DNA/RNA construct
accaggucgcgugcaacuatt

SEQ ID NO: 149 - DNA, 21 bp, Artificial Sequence
  Synthetic DNA/RNA construct
cucugggcgauucuggacctt

SEQ ID NO: 150 - DNA, 21 bp, Artificial Sequence
  Synthetic DNA/RNA construct
cuuuccgggucucuucugctt

SEQ ID NO: 151 - DNA, 21 bp, Artificial Sequence
  Synthetic DNA/RNA construct
cagaugugcacgaccgcggtt

SEQ ID NO: 152 - DNA, 21 bp, Artificial Sequence
  Synthetic DNA/RNA construct
cugggcgauucuggacccgtt

SEQ ID NO: 153 - DNA, 21 bp, Artificial Sequence
  Synthetic DNA/RNA construct
uucuccgguugcugaaggctt

SEQ ID NO: 154 - DNA, 21 bp, Artificial Sequence
  Synthetic DNA/RNA construct
uguaauucgacuccggugutt

SEQ ID NO: 155 - DNA, 21 bp, Artificial Sequence
  Synthetic DNA/RNA construct
uuacaccccgagaaaaguutt

SEQ ID NO: 156 - DNA, 21 bp, Artificial Sequence
  Synthetic DNA/RNA construct
gugugcagaggacucccaatt

SEQ ID NO: 157 - DNA, 21 bp, Artificial Sequence
  Synthetic DNA/RNA construct
gcucucggcuggugcccaatt

SEQ ID NO: 158 - DNA, 21 bp, Artificial Sequence
  Synthetic DNA/RNA construct
uuuccgggucucuucugcatt

SEQ ID NO: 159 - DNA, 21 bp, Artificial Sequence
  Synthetic DNA/RNA construct
cggcucugggcgauucuggtt

SEQ ID NO: 160 - DNA, 21 bp, Artificial Sequence
  Synthetic DNA/RNA construct
uugcugaaggcgcugcgcctt

SEQ ID NO: 161 - DNA, 21 bp, Artificial Sequence
  Synthetic DNA/RNA construct
ucacacaccaggucgcgugtt

SEQ ID NO: 162 - DNA, 21 bp, Artificial Sequence
  Synthetic DNA/RNA construct
ugggcgauucuggacccgctt

SEQ ID NO: 163 - DNA, 21 bp, Artificial Sequence
  Synthetic DNA/RNA construct
ccacucaaagcagagagaatt

SEQ ID NO: 164 - DNA, 21 bp, Artificial Sequence
  Synthetic DNA/RNA construct
gcggagccggcucucggcutt

SEQ ID NO: 165 - DNA, 21 bp, Artificial Sequence
  Synthetic DNA/RNA construct
cugacucgggucagaugugtt

SEQ ID NO: 166 - DNA, 21 bp, Artificial Sequence
  Synthetic DNA/RNA construct
ugcggagccggcucucggctt

SEQ ID NO: 167 - DNA, 21 bp, Artificial Sequence
  Synthetic DNA/RNA construct
ucucggcuggugcccaagctt

SEQ ID NO: 168 - DNA, 21 bp, Artificial Sequence
  Synthetic DNA/RNA construct
cgugcgagcagccaaucaatt

SEQ ID NO: 169 - DNA, 21 bp, Artificial Sequence
  Synthetic DNA/RNA construct
ugugugucaccaccccggctt

SEQ ID NO: 170 - DNA, 21 bp, Artificial Sequence
  Synthetic DNA/RNA construct
ucuucucagccaauggaggtt

SEQ ID NO: 171 - DNA, 21 bp, Artificial Sequence
  Synthetic DNA/RNA construct
ugcuucuccgguugcugaatt

SEQ ID NO: 172 - DNA, 21 bp, Artificial Sequence
  Synthetic DNA/RNA construct
cuucuccgguugcugaaggtt

SEQ ID NO: 173 - DNA, 21 bp, Artificial Sequence
  Synthetic DNA/RNA construct
agaggacucccaagccugctt

SEQ ID NO: 174 - DNA, 21 bp, Artificial Sequence
  Synthetic DNA/RNA construct
gaggacucccaagccugcutt

SEQ ID NO: 175 - DNA, 21 bp, Artificial Sequence
  Synthetic DNA/RNA construct
cucggcuggugcccaagcutt

SEQ ID NO: 176 - DNA, 21 bp, Artificial Sequence
  Synthetic DNA/RNA construct
cagaggacucccaagccugtt

SEQ ID NO: 177 - DNA, 21 bp, Artificial Sequence
  Synthetic DNA/RNA construct
ugacucgggucagaugugctt

SEQ ID NO: 178 - DNA, 21 bp, Artificial Sequence
  Synthetic DNA/RNA construct
cuccgguugcugaaggcgctt

SEQ ID NO: 179 - DNA, 21 bp, Artificial Sequence
  Synthetic DNA/RNA construct
cggagaucacacaccaggutt

SEQ ID NO: 180 - DNA, 21 bp, Artificial Sequence
  Synthetic DNA/RNA construct
auugcagggcuguaauucgtt

SEQ ID NO: 181 - DNA, 21 bp, Artificial Sequence
  Synthetic DNA/RNA construct
uugcagggcuguaauucgatt

SEQ ID NO: 182 - DNA, 21 bp, Artificial Sequence
  Synthetic DNA/RNA construct
cgagaaaaguucuugugugtt

SEQ ID NO: 183 - DNA, 21 bp, Artificial Sequence
  Synthetic DNA/RNA construct
ugugugcagaggacucccatt

SEQ ID NO: 184 - DNA, 21 bp, Artificial Sequence
  Synthetic DNA/RNA construct
agcuaggaucacagcuccctt

SEQ ID NO: 185 - DNA, 21 bp, Artificial Sequence
  Synthetic DNA/RNA construct
ucuccgguugcugaaggcgtt

SEQ ID NO: 186 - DNA, 21 bp, Artificial Sequence
  Synthetic DNA/RNA construct
aagcuaggaucacagcucctt

SEQ ID NO: 187 - DNA, 21 bp, Artificial Sequence
  Synthetic DNA/RNA construct
cagggcuguaauucgacuctt

SEQ ID NO: 188 - DNA, 21 bp, Artificial Sequence
  Synthetic DNA/RNA construct
ugaaggcgcugcgccccuutt

SEQ ID NO: 189 - DNA, 21 bp, Artificial Sequence
  Synthetic DNA/RNA construct
cuguaauucgacuccggugtt

SEQ ID NO: 190 - DNA, 21 bp, Artificial Sequence
  Synthetic DNA/RNA construct
aguucuugugugcagaggatt

SEQ ID NO: 191 - DNA, 21 bp, Artificial Sequence
  Synthetic DNA/RNA construct
cuggggugcgggagcggugtt

SEQ ID NO: 192 - DNA, 21 bp, Artificial Sequence
  Synthetic DNA/RNA construct
guucuugugugcagaggactt

SEQ ID NO: 193 - DNA, 21 bp, Artificial Sequence
  Synthetic DNA/RNA construct
gcugacucgggucagaugutt

SEQ ID NO: 194 - DNA, 21 bp, Artificial Sequence
  Synthetic DNA/RNA construct
caaagcagagagaaggccgtt

SEQ ID NO: 195 - DNA, 21 bp, Artificial Sequence
  Synthetic DNA/RNA construct
cacgcgccacucagucucctt

SEQ ID NO: 196 - DNA, 21 bp, Artificial Sequence
  Synthetic DNA/RNA construct
gcuucuccgguugcugaagtt

SEQ ID NO: 197 - DNA, 21 bp, Artificial Sequence
  Synthetic DNA/RNA construct
gcgggcggcucugggcgautt

SEQ ID NO: 198 - DNA, 21 bp, Artificial Sequence
  Synthetic DNA/RNA construct
acccgccaggcgcccagaatt

SEQ ID NO: 199 - DNA, 21 bp, Artificial Sequence
  Synthetic DNA/RNA Construct
ggcgcgugcgagcagccaatt

SEQ ID NO: 200 - DNA, 21 bp, Artificial Sequence
  Synthetic DNA/RNA construct
cucucggcuggugcccaagtt

SEQ ID NO: 201 - DNA, 21 bp, Artificial Sequence
  Synthetic DNA/RNA construct
agcucccacuucugaugcctt

SEQ ID NO: 202 - DNA, 21 bp, Artificial Sequence
  Synthetic DNA/RNA construct
gggcgauucuggacccgcctt

SEQ ID NO: 203 - DNA, 21 bp, Artificial Sequence
  Synthetic DNA/RNA construct
acagcucccacuucugaugtt

SEQ ID NO: 204 - DNA, 21 bp, Artificial Sequence
  Synthetic DNA/RNA construct
gaugugcacgaccgcgggctt

SEQ ID NO: 205 - DNA, 21 bp, Artificial Sequence
  Synthetic DNA/RNA construct
guuaauugcagggcuguaatt

SEQ ID NO: 206 - DNA, 21 bp, Artificial Sequence
  Synthetic DNA/RNA construct
uuccgggucucuucugcaatt

SEQ ID NO: 207 - DNA, 21 bp, Artificial Sequence
  Synthetic DNA/RNA construct
cugaaggcgcugcgccccutt

SEQ ID NO: 208 - DNA, 21 bp, Artificial Sequence
  Synthetic DNA/RNA construct
aaaguucuugugugcagagtt

SEQ ID NO: 209 - DNA, 21 bp, Artificial Sequence
  Synthetic DNA/RNA construct
cccacccacucggagaucatt

SEQ ID NO: 210 - DNA, 21 bp, Artificial Sequence
  Synthetic DNA/RNA construct
uccgggucucuucugcaactt

SEQ ID NO: 211 - DNA, 21 bp, Artificial Sequence
  Synthetic DNA/RNA construct
gaaaaguucuugugugcagtt

SEQ ID NO: 212 - DNA, 21 bp, Artificial Sequence
  Synthetic DNA/RNA construct
uggugcccaagcugacucgtt

SEQ ID NO: 213 - DNA, 21 bp, Artificial Sequence
  Synthetic DNA/RNA construct
ggcuggugcccaagcugactt

SEQ ID NO: 214 - DNA, 21 bp, Artificial Sequence
  Synthetic DNA/RNA construct
agggcuguaauucgacucctt

SEQ ID NO: 215 - DNA, 21 bp, Artificial Sequence
  Synthetic DNA/RNA construct
ucagaugugcacgaccgcgtt

SEQ ID NO: 216 - DNA, 21 bp, Artificial Sequence
  Synthetic DNA/RNA construct
acucggagaucacacaccatt

SEQ ID NO: 217 - DNA, 21 bp, Artificial Sequence
  Synthetic DNA/RNA construct
gggcaggcgcgugcgagcatt

SEQ ID NO: 218 - DNA, 21 bp, Artificial Sequence
  Synthetic DNA/RNA construct
cacuucugaugcccaagcatt

SEQ ID NO: 219 - DNA, 21 bp, Artificial Sequence
  Synthetic DNA/RNA construct
caggcgcccagaagccacutt

SEQ ID NO: 220 - DNA, 21 bp, Artificial Sequence
  Synthetic DNA/RNA construct
uucuugugugcagaggacutt

SEQ ID NO: 221 - DNA, 21 bp, Artificial Sequence
  Synthetic DNA/RNA construct
cccacucggagaucacacatt

SEQ ID NO: 222 - DNA, 21 bp, Artificial Sequence
  Synthetic DNA/RNA construct
ggacucccaagccugcucatt

SEQ ID NO: 223 - DNA, 21 bp, Artificial Sequence
  Synthetic DNA/RNA construct
cucgggucagaugugcacgtt

SEQ ID NO: 224 - DNA, 21 bp, Artificial Sequence
  Synthetic DNA/RNA construct
ucgggucagaugugcacgatt

SEQ ID NO: 225 - DNA, 21 bp, Artificial Sequence
  Synthetic DNA/RNA construct
aaagagccaaucagaagcutt

SEQ ID NO: 226 - DNA, 21 bp, Artificial Sequence
  Synthetic DNA/RNA construct
cgggucagaugugcacgactt

SEQ ID NO: 227 - DNA, 21 bp, Artificial Sequence
  Synthetic DNA/RNA construct
gaaggccgccuacccugugtt

SEQ ID NO: 228 - DNA, 21 bp, Artificial Sequence
  Synthetic DNA/RNA construct
cucggagaucacacaccagtt

SEQ ID NO: 229 - DNA, 21 bp, Artificial Sequence
  Synthetic DNA/RNA construct
uggggugcgggagcggugctt

SEQ ID NO: 230 - DNA, 21 bp, Artificial Sequence
  Synthetic DNA/RNA construct
caccccgagaaaaguucuutt

SEQ ID NO: 231 - DNA, 21 bp, Artificial Sequence
  Synthetic DNA/RNA construct
cccgagaaaaguucuugugtt

SEQ ID NO: 232 - DNA, 21 bp, Artificial Sequence
  Synthetic DNA/RNA construct
cucaaagcagagagaaggctt

SEQ ID NO: 233 - DNA, 21 bp, Artificial Sequence
  Synthetic DNA/RNA construct
agcggugcggagccggcuctt

SEQ ID NO: 234 - DNA, 21 bp, Artificial Sequence
  Synthetic DNA/RNA construct
cuccucgcgccggucgcgutt

SEQ ID NO: 235 - DNA, 21 bp, Artificial Sequence
  Synthetic DNA/RNA construct
uucugaugcccaagcauuutt

SEQ ID NO: 236 - DNA, 21 bp, Artificial Sequence
  Synthetic DNA/RNA construct
ccacuucugaugcccaagctt

SEQ ID NO: 237 - DNA, 21 bp, Artificial Sequence
  Synthetic DNA/RNA construct
ugcacgaccgcgggcggcutt

SEQ ID NO: 238 - DNA, 21 bp, Artificial Sequence
  Synthetic DNA/RNA construct
gggucagaugugcacgacctt

SEQ ID NO: 239 - DNA, 21 bp, Artificial Sequence
  Synthetic DNA/RNA construct
augcuucuccgguugcugatt

SEQ ID NO: 240 - DNA, 21 bp, Artificial Sequence
  Synthetic DNA/RNA construct
ggcggcucugggcgauucutt

SEQ ID NO: 241 - DNA, 21 bp, Artificial Sequence
  Synthetic DNA/RNA construct
caagcuaggaucacagcuctt

SEQ ID NO: 242 - DNA, 21 bp, Artificial Sequence
  Synthetic DNA/RNA construct
gugcaacuaugcuucuccgtt

SEQ ID NO: 243 - DNA, 21 bp, Artificial Sequence
  Synthetic DNA/RNA construct
ccaggucgcgugcaacuautt

SEQ ID NO: 244 - DNA, 21 bp, Artificial Sequence
  Synthetic DNA/RNA construct
uuuacaccccgagaaaagutt

SEQ ID NO: 245 - DNA, 21 bp, Artificial Sequence
  Synthetic DNA/RNA construct
cccgccaggcgcccagaagtt

SEQ ID NO: 246 - DNA, 21 bp, Artificial Sequence
  Synthetic DNA/RNA construct
gucaccaccccggcggcugtt

SEQ ID NO: 247 - DNA, 21 bp, Artificial Sequence
  Synthetic DNA/RNA construct
ccaagcuaggaucacagcutt

SEQ ID NO: 248 - DNA, 21 bp, Artificial Sequence
  Synthetic DNA/RNA construct
agaucacacaccaggucgctt

SEQ ID NO: 249 - DNA, 21 bp, Artificial Sequence
  Synthetic DNA/RNA construct
aggccgccuacccugugcutt

SEQ ID NO: 250 - DNA, 21 bp, Artificial Sequence
  Synthetic DNA/RNA construct
uucuggacccgccaggcgctt

SEQ ID NO: 251 - DNA, 21 bp, Artificial Sequence
  Synthetic DNA/RNA construct
cagcucccacuucugaugctt

SEQ ID NO: 252 - DNA, 21 bp, Artificial Sequence
  Synthetic DNA/RNA construct
gggcggcucugggcgauuctt

SEQ ID NO: 253 - DNA, 21 bp, Artificial Sequence
  Synthetic DNA/RNA construct
ugcugaaggcgcugcgccctt

SEQ ID NO: 254 - DNA, 21 bp, Artificial Sequence
  Synthetic DNA/RNA construct
uguuaauugcagggcuguatt

SEQ ID NO: 255 - DNA, 21 bp, Artificial Sequence
  Synthetic DNA/RNA construct
uucgucagauucauauguutt

SEQ ID NO: 256 - DNA, 21 bp, Artificial Sequence
  Synthetic DNA/RNA construct
gcuggggugcgggagcggutt

SEQ ID NO: 257 - DNA, 21 bp, Artificial Sequence
  Synthetic DNA/RNA construct
ggcacgcgccacucagucutt

SEQ ID NO: 258 - DNA, 21 bp, Artificial Sequence
  Synthetic DNA/RNA construct
aggucgcgugcaacuaugctt

SEQ ID NO: 259 - DNA, 21 bp, Artificial Sequence
  Synthetic DNA/RNA construct
gacucgggucagaugugcatt

SEQ ID NO: 260 - DNA, 21 bp, Artificial Sequence
  Synthetic DNA/RNA construct
cgggcggcucugggcgauutt

SEQ ID NO: 261 - DNA, 21 bp, Artificial Sequence
  Synthetic DNA/RNA construct
gugcccaagcugacucgggtt

SEQ ID NO: 262 - DNA, 21 bp, Artificial Sequence
  Synthetic DNA/RNA construct
ggcucucggcuggugcccatt

SEQ ID NO: 263 - DNA, 21 bp, Artificial Sequence
  Synthetic DNA/RNA construct
uuuuacaccccgagaaaagtt

SEQ ID NO: 264 - DNA, 21 bp, Artificial Sequence
  Synthetic DNA/RNA construct
cccagaagccacucaaagctt

SEQ ID NO: 265 - DNA, 21 bp, Artificial Sequence
  Synthetic DNA/RNA construct
ugcagggcuguaauucgactt

SEQ ID NO: 266 - DNA, 21 bp, Artificial Sequence
  Synthetic DNA/RNA construct
gucagaugugcacgaccgctt

SEQ ID NO: 267 - DNA, 21 bp, Artificial Sequence
  Synthetic DNA/RNA construct
uaauugcagggcuguaauutt

SEQ ID NO: 268 - DNA, 21 bp, Artificial Sequence
  Synthetic DNA/RNA construct
cgggucucuucugcaacgutt

SEQ ID NO: 269 - DNA, 21 bp, Artificial Sequence
  Synthetic DNA/RNA construct
ucuggacccgccaggcgcctt

SEQ ID NO: 270 - DNA, 21 bp, Artificial Sequence
  Synthetic DNA/RNA construct
cacacaccaggucgcgugctt

SEQ ID NO: 271 - DNA, 21 bp, Artificial Sequence
  Synthetic DNA/RNA construct
acacaccaggucgcgugcatt

SEQ ID NO: 272 - DNA, 21 bp, Artificial Sequence
  Synthetic DNA/RNA construct
ugugucaccaccccggcggtt

SEQ ID NO: 273 - DNA, 21 bp, Artificial Sequence
  Synthetic DNA/RNA construct
gcgagcagccaaucaaagatt

SEQ ID NO: 274 - DNA, 21 bp, Artificial Sequence
  Synthetic DNA/RNA construct
uaauucgacuccggugugutt

SEQ ID NO: 275 - DNA, 21 bp, Artificial Sequence
  Synthetic DNA/RNA construct
cuucugaugcccaagcauutt

SEQ ID NO: 276 - DNA, 21 bp, Artificial Sequence
  Synthetic DNA/RNA construct
agcagccaaucaaagaguutt

SEQ ID NO: 277 - DNA, 21 bp, Artificial Sequence
  Synthetic DNA/RNA construct
ucugggcgauucuggaccctt

SEQ ID NO: 278 - DNA, 21 bp, Artificial Sequence
  Synthetic DNA/RNA construct
guaauucgacuccggugugtt

SEQ ID NO: 279 - DNA, 21 bp, Artificial Sequence
  Synthetic DNA/RNA construct
ucuugugugcagaggacuctt

SEQ ID NO: 280 - DNA, 19 bp, Artificial Sequence
  Active target sequence
gcggccttctctctgcttt

SEQ ID NO: 281 - DNA, 19 bp, Artificial Sequence
  Active target sequence
cagaagagacccggaaagg

SEQ ID NO: 282 - DNA, 19 bp, Artificial Sequence
  Active target sequence
ccggagaagcatagttgca

SEQ ID NO: 283 - DNA, 19 bp, Artificial Sequence
  Active target sequence
cgcgacctggtgtgtgatc

SEQ ID NO: 284 - DNA, 19 bp, Artificial Sequence
  Active target sequence
gagcaggcttgggagtcct

SEQ ID NO: 285 - DNA, 19 bp, Artificial Sequence
  Active target sequence
tagttgcacgcgacctggt

SEQ ID NO: 286 - DNA, 19 bp, Artificial Sequence
  Active target sequence
ggtccagaatcgcccagag

SEQ ID NO: 287 - DNA, 19 bp, Artificial Sequence
  Active target sequence
gcagaagagacccggaaag

SEQ ID NO: 288 - DNA, 19 bp, Artificial Sequence
  Active target sequence
ccgcggtcgtgcacatctg

SEQ ID NO: 289 - DNA, 19 bp, Artificial Sequence
  Active target sequence
cgggtccagaatcgcccag

SEQ ID NO: 290 - DNA, 19 bp, Artificial Sequence
  Active target sequence
gccttcagcaaccggagaa

SEQ ID NO: 291 - DNA, 19 bp, Artificial Sequence
  Active target sequence
acaccggagtcgaattaca

SEQ ID NO: 292 - DNA, 19 bp, Artificial Sequence
  Active target sequence
aacttttctcggggtgtaa

SEQ ID NO: 293 - DNA, 19 bp, Artificial Sequence
  Active target sequence
ttgggagtcctctgcacac

SEQ ID NO: 294 - DNA, 19 bp, Artificial Sequence
  Active target sequence
ttgggcaccagccgagagc

SEQ ID NO: 295 - DNA, 19 bp, Artificial Sequence
  Active target sequence
tgcagaagagacccggaaa

SEQ ID NO: 296 - DNA, 19 bp, Artificial Sequence
  Active target sequence
ccagaatcgcccagagccg

SEQ ID NO: 297 - DNA, 19 bp, Artificial Sequence
  Active target sequence
ggcgcagcgccttcagcaa

SEQ ID NO: 298 - DNA, 19 bp, Artificial Sequence
  Active target sequence
cacgcgacctggtgtgtga

SEQ ID NO: 299 - DNA, 19 bp, Artificial Sequence
  Active target sequence
gcgggtccagaatcgccca

SEQ ID NO: 300 - DNA, 19 bp, Artificial Sequence
  Active target sequence
ttctctctgctttgagtgg

SEQ ID NO: 301 - DNA, 19 bp, Artificial Sequence
  Active target sequence
agccgagagccggctccgc

SEQ ID NO: 302 - DNA, 19 bp, Artificial Sequence
  Active target sequence
cacatctgacccgagtcag

SEQ ID NO: 303 - DNA, 19 bp, Artificial Sequence
  Active target sequence
gccgagagccggctccgca

SEQ ID NO: 304 - DNA, 19 bp, Artificial Sequence
  Active target sequence
gcttgggcaccagccgaga

SEQ ID NO: 305 - DNA, 19 bp, Artificial Sequence
  Active target sequence
ttgattggctgctcgcacg

SEQ ID NO: 306 - DNA, 19 bp, Artificial Sequence
  Active target sequence
gccggggtggtgacacaca

SEQ ID NO: 307 - DNA, 19 bp, Artificial Sequence
  Active target sequence
cctccattggctgagaaga

SEQ ID NO: 308 - DNA, 19 bp, Artificial Sequence
  Active target sequence
ttcagcaaccggagaagca

SEQ ID NO: 309 - DNA, 19 bp, Artificial Sequence
  Active target sequence
ccttcagcaaccggagaag

SEQ ID NO: 310 - DNA, 19 bp, Artificial Sequence
  Active target sequence
gcaggcttgggagtcctct

SEQ ID NO: 311 - DNA, 19 bp, Artificial Sequence
  Active target sequence
agcaggcttgggagtcctc

SEQ ID NO: 312 - DNA, 19 bp, Artificial Sequence
  Active target sequence
agcttgggcaccagccgag

SEQ ID NO: 313 - DNA, 19 bp, Artificial Sequence
  Active target sequence
caggcttgggagtcctctg

SEQ ID NO: 314 - DNA, 19 bp, Artificial Sequence
  Active target sequence
gcacatctgacccgagtca

SEQ ID NO: 315 - DNA, 19 bp, Artificial Sequence
  Active target sequence
gcgccttcagcaaccggag

SEQ ID NO: 316 - DNA, 19 bp, Artificial Sequence
  Active target sequence
acctggtgtgtgatctccg

SEQ ID NO: 317 - DNA, 19 bp, Artificial Sequence
  Active target sequence
cgaattacagccctgcaat

SEQ ID NO: 318 - DNA, 19 bp, Artificial Sequence
  Active target sequence
tcgaattacagccctgcaa

SEQ ID NO: 319 - DNA, 19 bp, Artificial Sequence
  Active target sequence
cacacaagaacttttctcg

SEQ ID NO: 320 - DNA, 19 bp, Artificial Sequence
  Active target sequence
tgggagtcctctgcacaca

SEQ ID NO: 321 - DNA, 19 bp, Artificial Sequence
  Active target sequence
gggagctgtgatcctagct

SEQ ID NO: 322 - DNA, 19 bp, Artificial Sequence
  Active target sequence
cgccttcagcaaccggaga

SEQ ID NO: 323 - DNA, 19 bp, Artificial Sequence
  Active target sequence
ggagctgtgatcctagctt

SEQ ID NO: 324 - DNA, 19 bp, Artificial Sequence
  Active target sequence
gagtcgaattacagccctg

SEQ ID NO: 325 - DNA, 19 bp, Artificial Sequence
  Active target sequence
aaggggcgcagcgccttca

SEQ ID NO: 326 - DNA, 19 bp, Artificial Sequence
  Active target sequence
caccggagtcgaattacag

SEQ ID NO: 327 - DNA, 19 bp, Artificial Sequence
  Active target sequence
tcctctgcacacaagaact

SEQ ID NO: 328 - DNA, 19 bp, Artificial Sequence
  Active target sequence
caccgctcccgcaccccag

SEQ ID NO: 329 - DNA, 19 bp, Artificial Sequence
  Active target sequence
gtcctctgcacacaagaac

SEQ ID NO: 330 - DNA, 19 bp, Artificial Sequence
  Active target sequence
acatctgacccgagtcagc

SEQ ID NO: 331 - DNA, 19 bp, Artificial Sequence
  Active target sequence
cggccttctctctgctttg

SEQ ID NO: 332 - DNA, 19 bp, Artificial Sequence
  Active target sequence
ggagactgagtggcgcgtg

SEQ ID NO: 333 - DNA, 19 bp, Artificial Sequence
  Active target sequence
cttcagcaaccggagaagc

SEQ ID NO: 334 - DNA, 19 bp, Artificial Sequence
  Active target sequence
atcgcccagagccgcccgc

SEQ ID NO: 335 - DNA, 19 bp, Artificial Sequence
  Active target sequence
ttctgggcgcctggcgggt

SEQ ID NO: 336 - DNA, 19 bp, Artificial Sequence
  Active target sequence
ttggctgctcgcacgcgcc

SEQ ID NO: 337 - DNA, 19 bp, Artificial Sequence
  Active target sequence
cttgggcaccagccgagag

SEQ ID NO: 338 - DNA, 19 bp, Artificial Sequence
  Active target sequence
ggcatcagaagtgggagct

SEQ ID NO: 339 - DNA, 19 bp, Artificial Sequence
  Active target sequence
ggcgggtccagaatcgccc

SEQ ID NO: 340 - DNA, 19 bp, Artificial Sequence
  Active target sequence
catcagaagtgggagctgt

SEQ ID NO: 341 - DNA, 19 bp, Artificial Sequence
  Active target sequence
gcccgcggtcgtgcacatc

SEQ ID NO: 342 - DNA, 19 bp, Artificial Sequence
  Active target sequence
ttacagccctgcaattaac

SEQ ID NO: 343 - DNA, 19 bp, Artificial Sequence
  Active target sequence
ttgcagaagagacccggaa

SEQ ID NO: 344 - DNA, 19 bp, Artificial Sequence
  Active target sequence
aggggcgcagcgccttcag

SEQ ID NO: 345 - DNA, 19 bp, Artificial Sequence
  Active target sequence
ctctgcacacaagaacttt

SEQ ID NO: 346 - DNA, 19 bp, Artificial Sequence
  Active target sequence
tgatctccgagtgggtggg

SEQ ID NO: 347 - DNA, 19 bp, Artificial Sequence
  Active target sequence
gttgcagaagagacccgga

SEQ ID NO: 348 - DNA, 19 bp, Artificial Sequence
  Active target sequence
ctgcacacaagaacttttc

SEQ ID NO: 349 - DNA, 19 bp, Artificial Sequence
  Active target sequence
cgagtcagcttgggcacca

SEQ ID NO: 350 - DNA, 19 bp, Artificial Sequence
  Active target sequence
gtcagcttgggcaccagcc

SEQ ID NO: 351 - DNA, 19 bp, Artificial Sequence
  Active target sequence
ggagtcgaattacagccct

SEQ ID NO: 352 - DNA, 19 bp, Artificial Sequence
  Active target sequence
cgcggtcgtgcacatctga

SEQ ID NO: 353 - DNA, 19 bp, Artificial Sequence
  Active target sequence
tggtgtgtgatctccgagt

SEQ ID NO: 354 - DNA, 19 bp, Artificial Sequence
  Active target sequence
tgctcgcacgcgcctgccc

SEQ ID NO: 355 - DNA, 19 bp, Artificial Sequence
  Active target sequence
tgcttgggcatcagaagtg

SEQ ID NO: 356 - DNA, 19 bp, Artificial Sequence
  Active target sequence
agtggcttctgggcgcctg

SEQ ID NO: 357 - DNA, 19 bp, Artificial Sequence
  Active target sequence
agtcctctgcacacaagaa

SEQ ID NO: 358 - DNA, 19 bp, Artificial Sequence
  Active target sequence
tgtgtgatctccgagtggg

SEQ ID NO: 359 - DNA, 19 bp, Artificial Sequence
  Active target sequence
tgagcaggcttgggagtcc

SEQ ID NO: 360 - DNA, 19 bp, Artificial Sequence
  Active target sequence
cgtgcacatctgacccgag

SEQ ID NO: 361 - DNA, 19 bp, Artificial Sequence
  Active target sequence
tcgtgcacatctgacccga

SEQ ID NO: 362 - DNA, 19 bp, Artificial Sequence
  Active target sequence
agcttctgattggctcttt

SEQ ID NO: 363 - DNA, 19 bp, Artificial Sequence
  Active target sequence
gtcgtgcacatctgacccg

SEQ ID NO: 364 - DNA, 19 bp, Artificial Sequence
  Active target sequence
cacagggtaggcggccttc

SEQ ID NO: 365 - DNA, 19 bp, Artificial Sequence
  Active target sequence
ctggtgtgtgatctccgag

SEQ ID NO: 366 - DNA, 19 bp, Artificial Sequence
  Active target sequence
gcaccgctcccgcacccca

SEQ ID NO: 367 - DNA, 19 bp, Artificial Sequence
  Active target sequence
aagaacttttctcggggtg

SEQ ID NO: 368 - DNA, 19 bp, Artificial Sequence
  Active target sequence
cacaagaacttttctcggg

SEQ ID NO: 369 - DNA, 19 bp, Artificial Sequence
  Active target sequence
gccttctctctgctttgag

SEQ ID NO: 370 - DNA, 19 bp, Artificial Sequence
  Active target sequence
gagccggctccgcaccgct

SEQ ID NO: 371 - DNA, 19 bp, Artificial Sequence
  Active target sequence
acgcgaccggcgcgaggag

SEQ ID NO: 372 - DNA, 19 bp, Artificial Sequence
  Active target sequence
aaatgcttgggcatcagaa

SEQ ID NO: 373 - DNA, 19 bp, Artificial Sequence
  Active target sequence
gcttgggcatcagaagtgg

SEQ ID NO: 374 - DNA, 19 bp, Artificial Sequence
  Active target sequence
agccgcccgcggtcgtgca

SEQ ID NO: 375 - DNA, 19 bp, Artificial Sequence
  Active target sequence
ggtcgtgcacatctgaccc

SEQ ID NO: 376 - DNA, 19 bp, Artificial Sequence
  Active target sequence
tcagcaaccggagaagcat

SEQ ID NO: 377 - DNA, 19 bp, Artificial Sequence
  Active target sequence
agaatcgcccagagccgcc

SEQ ID NO: 378 - DNA, 19 bp, Artificial Sequence
  Active target sequence
gagctgtgatcctagcttg

SEQ ID NO: 379 - DNA, 19 bp, Artificial Sequence
  Active target sequence
cggagaagcatagttgcac

SEQ ID NO: 380 - DNA, 19 bp, Artificial Sequence
  Active target sequence
atagttgcacgcgacctgg

SEQ ID NO: 381 - DNA, 19 bp, Artificial Sequence
  Active target sequence
acttttctcggggtgtaaa

SEQ ID NO: 382 - DNA, 19 bp, Artificial Sequence
  Active target sequence
cttctgggcgcctggcggg

SEQ ID NO: 383 - DNA, 19 bp, Artificial Sequence
  Active target sequence
cagccgccggggtggtgac

SEQ ID NO: 384 - DNA, 19 bp, Artificial Sequence
  Active target sequence
agctgtgatcctagcttgg

SEQ ID NO: 385 - DNA, 19 bp, Artificial Sequence
  Active target sequence
gcgacctggtgtgtgatct

SEQ ID NO: 386 - DNA, 19 bp, Artificial Sequence
  Active target sequence
agcacagggtaggcggcct

SEQ ID NO: 387 - DNA, 19 bp, Artificial Sequence
  Active target sequence
gcgcctggcgggtccagaa

SEQ ID NO: 388 - DNA, 19 bp, Artificial Sequence
  Active target sequence
gcatcagaagtgggagctg

SEQ ID NO: 389 - DNA, 19 bp, Artificial Sequence
  Active target sequence
gaatcgcccagagccgccc

SEQ ID NO: 390 - DNA, 19 bp, Artificial Sequence
  Active target sequence
gggcgcagcgccttcagca

SEQ ID NO: 391 - DNA, 19 bp, Artificial Sequence
  Active target sequence
tacagccctgcaattaaca

SEQ ID NO: 392 - DNA, 19 bp, Artificial Sequence
  Active target sequence
aacatatgaatctgacgaa

SEQ ID NO: 393 - DNA, 19 bp, Artificial Sequence
  Active target sequence
accgctcccgcaccccagc

SEQ ID NO: 394 - DNA, 19 bp, Artificial Sequence
  Active target sequence
agactgagtggcgcgtgcc

SEQ ID NO: 395 - DNA, 19 bp, Artificial Sequence
  Active target sequence
gcatagttgcacgcgacct

SEQ ID NO: 396 - DNA, 19 bp, Artificial Sequence
  Active target sequence
tgcacatctgacccgagtc

SEQ ID NO: 397 - DNA, 19 bp, Artificial Sequence
  Active target sequence
aatcgcccagagccgcccg

SEQ ID NO: 398 - DNA, 19 bp, Artificial Sequence
  Active target sequence
cccgagtcagcttgggcac

SEQ ID NO: 399 - DNA, 19 bp, Artificial Sequence
  Active target sequence
tgggcaccagccgagagcc

SEQ ID NO: 400 - DNA, 19 bp, Artificial Sequence
  Active target sequence
cttttctcggggtgtaaaa

SEQ ID NO: 401 - DNA, 19 bp, Artificial Sequence
  Active target sequence
gctttgagtggcttctggg

SEQ ID NO: 402 - DNA, 19 bp, Artificial Sequence
  Active target sequence
gtcgaattacagccctgca

SEQ ID NO: 403 - DNA, 19 bp, Artificial Sequence
  Active target sequence
gcggtcgtgcacatctgac

SEQ ID NO: 404 - DNA, 19 bp, Artificial Sequence
  Active target sequence
aattacagccctgcaatta

SEQ ID NO: 405 - DNA, 19 bp, Artificial Sequence
  Active target sequence
acgttgcagaagagacccg

SEQ ID NO: 406 - DNA, 19 bp, Artificial Sequence
  Active target sequence
ggcgcctggcgggtccaga

SEQ ID NO: 407 - DNA, 19 bp, Artificial Sequence
  Active target sequence
gcacgcgacctggtgtgtg

SEQ ID NO: 408 - DNA, 19 bp, Artificial Sequence
  Active target sequence
tgcacgcgacctggtgtgt

SEQ ID NO: 409 - DNA, 19 bp, Artificial Sequence
  Active target sequence
ccgccggggtggtgacaca

SEQ ID NO: 410 - DNA, 19 bp, Artificial Sequence
  Active target sequence
tctttgattggctgctcgc

SEQ ID NO: 411 - DNA, 19 bp, Artificial Sequence
  Active target sequence
acacaccggagtcgaatta

SEQ ID NO: 412 - DNA, 19 bp, Artificial Sequence
  Active target sequence
aatgcttgggcatcagaag

SEQ ID NO: 413 - DNA, 19 bp, Artificial Sequence
  Active target sequence
aactctttgattggctgct

SEQ ID NO: 414 - DNA, 19 bp, Artificial Sequence
  Active target sequence
gggtccagaatcgcccaga

SEQ ID NO: 415 - DNA, 19 bp, Artificial Sequence
  Active target sequence
cacaccggagtcgaattac

SEQ ID NO: 416 - DNA, 19 bp, Artificial Sequence
  Active target sequence
gagtcctctgcacacaaga

SEQ ID NO: 417 - DNA, 20 bp, Artificial Sequence
  Primer sequence
cccttccagcaaacaggtga

SEQ ID NO: 418 - DNA, 21 bp, Artificial Sequence
  Primer sequence
gaacgacgggataacattgcg

SEQ ID NO: 419 - DNA, 21 bp, Artificial Sequence
  Primer sequence
atggacaggactgaacgtctt

SEQ ID NO: 420 - DNA, 20 bp, Artificial Sequence
  Primer sequence
tccagcaggtcagcaaagaa

SEQ ID NO: 421 - DNA, 21 bp, Artificial Sequence
  Synthetic DNA/RNA construct
acuacugagugacaguagatt

SEQ ID NO: 422 - DNA, 21 bp, Artificial Sequence
  Synthetic DNA/RNA construct
ucuacugucacucaguagutt

SEQ ID NO: 423 - DNA, 21 bp, Artificial Sequence
  Synthetic DNA/RNA construct
gcucugcacuucucgacuutt

SEQ ID NO: 424 - DNA, 21 bp, Artificial Sequence
  Synthetic DNA/RNA construct
aagucgagaagugcagagctt

(19) United States
(12) Patent Application Publication
(10) Pub. No.: US 2022/0313721 A1
(43) Pub. Date: Oct. 6, 2022

(54) OLIGOMERIC NUCLEIC ACID MOLECULE ACTIVATING ATOH1 GENE AND USE THEREOF

(71) Applicant: Ractigen Therapeutics, Nantong City, Jiangsu (CN)

(72) Inventors: Longcheng LI, Nantong City, Jiangsu (CN); Moorim KANG, Nantong City, Jiangsu (CN)

(21) Appl. No.: 17/597,281

(22) PCT Filed: Jul. 3, 2020
(86) PCT No.: PCT/CN2020/100093

§ 371 Date: Dec. 30, 2021

(30) Foreign Application Priority Data
Jul. 4, 2019 (CN) 201910598221.6

(51) Int. Cl. A61K 31/7088; A61K 48/00; A61P 27/16

(52) U.S. Cl. CPC A61K 31/7088; A61K 48/00; A61P 27/16

(57) ABSTRACT

The present invention relates to oligomeric nucleic acid molecules and uses thereof for treating hearing loss. The present invention relates to small activating nucleic acid molecules for treating hearing loss. The small activating nucleic acid molecules of the present invention can be double-stranded or single-stranded RNA molecules targeting the promoter region of an ATOH1 gene comprising a first nucleic acid strand and a second nucleic acid strand. The double-stranded RNA molecule targeting the promoter region of the ATOH1 gene comprises two nucleic acid strands of 16 to 35 nucleotides in length, wherein one nucleic acid strand has at least 75% homology or complementarity to a target selected from the promoter region of the ATOH1 gene. The present invention also relates to pharmaceutical compositions comprising the small activating nucleic acid molecules and optionally, a pharmaceutically acceptable carrier, and methods for upregulating the expression of the ATOH1 gene and ATOH1 protein in a cell and treating diseases or conditions, related to insufficient or decreased expression of the ATOH1 gene by using the small activating nucleic acid molecules, or the pharmaceutical composition.

### TECHNICAL FIELD

The present invention belongs to the technical field of nucleic acids, particularly as it relates to oligomeric nucleic acid molecules associated with gene activation, e.g., small activating nucleic acid molecules, uses of small activating nucleic acid molecules in activating or upregulating the transcription of the ATOH1 gene, and uses thereof for treating diseases or conditions related to ATOH1 protein deficiency or insufficiency, such as for uses in treating hearing loss.

### BACKGROUND

Hearing loss is a serious global disease affecting 360 million people worldwide. Hearing loss not only affects the quality of life of patients, but is also a heavy burden on the medical system. An effective treatment method for hearing loss is lacking and there is an urgent need to develop new methods of treatment (WHO, 2013).

Hearing loss is a manifestation of hearing dysfunction and is clinically classified into three types: conductive, sensorineural, and mixed hearing loss. In conductive hearing loss, lesions are found in outer or middle ear causing an obstruction to the transmission of sound waves to the inner ear. Sensorineural hearing loss is the most common type of hearing loss and caused by the loss of hair cells in the inner cochlea (Liberman 1990). Any factor that causes both conductive and sensorineural hearing loss is referred to as mixed hearing loss. The human inner ear contains about 75,000 sensory hair cells which detect sound and motion through the mechanically-sensitive stereocilia bundle which are mechanical sensors that convert motion signals into electrical signals. The sound transmission mechanism is the adaptive amplification of sound by the inner ear through the outer hair cells and synaptic chemical transmission of inner hair cells, converting mechanical vibration of sound into electrical impulses on auditory nerve fibers. The electrical impulses are further processed, encoded, and interpreted by auditory brainstem and auditory cortex, finally presenting auditory imagery to external acoustic vibrations at a cognitive level.

The factors influencing hearing loss are classified into congenital and acquired hearing losses. Congenital factors mainly refer to hearing loss of a newborn at or shortly after birth, which can be caused by infection or acquired disease of a pregnant woman during pregnancy, improper use of special drugs, the occurrence of complications during childbirth, and the like. Acquired factors may occur at any age, including infection, head and ear trauma, prolonged exposure to environments with excessive noise and ototoxic drugs, and aging which result in the deterioration of sensory hair cells of cochlea (Gurtler and Lalwani 2002). In normal populations, men have hearing loss beginning at about age 45, and women a few years later. Over 40% of the population over 50 years of age and 70% of the population over 70 years of age have clinically significant hearing loss. As a result of an aging of population, the incidence of deafness in the elderly has also increased. In addition, the loss of hair cells in the vestibular epithelial cells of the inner ear also leads to balance dysfunction, causing dizziness and vertigo.

The ATOH1 genes, also known as ATH1, HATH1, MATH-1 and bHLHa14, encode atonal bHLH transcription factor 1 proteins, and belong to members of the basic helix-loop-helix (bHLH) transcription factor family. ATOH1 has a positive regulation effect in regenerative processes of inner ear hair cell morphology and function of mammals (Zheng and Gao 2000). ATOH1 was the first gene found to be expressed in the development of the inner ear (Bermingham et al. 1999), and it regulates the development of nerve cells, such as the spinal cord, inner ear hair cells, cerebellar neurons, proprioceptive system neurons, and non-nerve cells. Hair cell regeneration is important during the development of the inner ear and ATOH1 promotes hair cell differentiation and regeneration.

Therefore, overexpressing ATOH1 can provide a new therapy for hearing loss by promoting the regeneration of hair cells. In addition, ATOH1 also functions as a tumor suppressor gene. ATOH1 is a definite tumor suppressor gene in colorectal cancer, and overexpression of ATOH1 can suppress tumor cell proliferation and growth of transplanted tumors (Kazanjian and Shroyer 2011).

Vector-mediated, e.g., viral and liposomal,) ATOH1 gene therapy has been shown to promote hair cell regeneration and improve hearing in animal experiments (Richardson and Atkinson 2015). Some investigators have induced hair cells to differentiate and regenerate by mediating the overexpression of ATOH1 prior to the collapse of the Organ of Corti (Kawamoto et al. 2003; Gubbels et al. 2008). The Raphael Group at The University of Michigan reported that aminoglycoside antibiotics caused the regeneration of hair cells with partial restoration of auditory function after overexpressing ATOH1 in the inner ear of fully deaf domesticated guinea pigs (Izumikawa et al. 2005). In neonatal mice the overexpression of ATOH1 can convert supporting cells into hair cells which can promote the entry of other cells, e.g., intestinal cells, into a differentiated state (Aragaki et al. 2008), and reduce the proliferation of colon cancer cells (Leow, Polakis, and Gao 2005).

This gene delivery method usually requires vectors, and the currently used gene transfection vectors are viruses and liposomes. The current gene therapy product, CGF166, a recombinant adenovirus-mediated ATOH1 gene therapy, is being tested in Phase I Clinical Trial to treat hearing loss (Novartis, ClinicalTrials.gov Identifier: NCT02132130). Although the expression levels of the viral-gene therapy system is high, problems with vector safety and immune response cannot be completely overcome and the applications of a viral-gene therapy system for use in a human presents certain challenges. Therefore, the development of new treatment method is of particular importance.

### SUMMARY

One objective of the present invention is to provide a small activating nucleic acid molecule based on RNA activation process, which can increase the expression of ATOH1 protein by activating or upregulating the transcription of ATOH1 to promote the regeneration of inner ear hair cells, restore hearing, or treat a disease or condition related to ATOH1 protein deficiency or insufficiency, such as tumor formation.

Another objective of the present invention is to provide a composition or a formulation comprising a small activating nucleic acid molecule.

Another objective of the present invention is to provide a use of a small activating nucleic acid molecule or a composition or formulation comprising the same in preparing a drug for activating or upregulating the expression of ATOH1 in a cell.

Yet another objective of the present invention is to provide a method for activating or upregulating the expression of ATOH1 in a cell.

Yet another objective of the present invention is to provide a use of a small activating nucleic acid molecule or a composition or formulation thereof in the preparation of a drug for treating a disease or a condition related to ATOH1 protein deficiency or insufficiency, or in a method of treating a disease or condition such as hearing loss related to ATOH1 protein deficiency or insufficiency.

Still yet another objective of the present invention is to provide an isolated small activating nucleic acid molecule target site of the ATOH1 gene, wherein the target site comprises or is selected from any continuous sequence of 16 to 35 nucleotides in length in any of sequences set forth in SEQ ID NO: 2 to SEQ ID NO: 5.

### TECHNICAL SOLUTION

In one aspect of the present invention, a small activating nucleic acid molecule (e.g., a small activating RNA saRNA) activating or upregulating the expression of ATOH1 in a cell is provided, wherein one strand of a small activating nucleic acid molecule has at least 75% homology or complementarity to any nucleic acid sequence of 16 to 35 nucleotides in length in the promoter region of ATOH1, thereby activating or upregulating the expression of the gene, wherein the promoter region comprises 637 nucleotides upstream of a transcription start site. Specifically, one strand of a small activating nucleic acid molecule comprises or is selected from a nucleic acid sequence having at least 75% (e.g., at least about 79%, about 80%, about 85%, about 90%, about 95%, about 99% or 100%) homology or complementarity to a continuous sequence of 16 to 35 nucleotides in length in positions −578 to −386 (hotspot 1, SEQ ID NO: 2), positions −339 to −291 (hotspot 2, SEQ ID NO: 3), positions −162 to −86 (hotspot 3, SEQ ID NO: 4) or positions −55 to −34 (hotspot 4, SEQ ID NO: 5) upstream of the transcription start site of the ATOH1 gene promoter. More specifically, one strand of a small activating nucleic acid molecule of the present invention has at least 75% (e.g., at least about 79%, about 80%, about 85%, about 90%, about 95%, about 99% or about 100%) homology or complementarity to any nucleotide sequence selected from SEQ ID NOs: 280-416. In one specific embodiment, one strand of a small activating nucleic acid molecule of the present invention comprises a nucleic acid sequence having at least 75% (e.g., at least about 79%, about 80%, about 85%, about 90%, about 95%, about 99% or about 100%) homology or complementarity to any nucleotide sequence selected from SEQ ID NOs: 280-416. In another embodiment, one strand of a small activating nucleic acid molecule of the present invention consists of a nucleic acid sequence having at least 75% (e.g., at least about 79%, about 80%, about 85%, about 90%, about 95%, about 99% or about 100%) homology or complementarity to any nucleotide sequence selected from SEQ ID NOs: 280-416. In yet another embodiment, one strand of a small activating nucleic acid molecule of the present invention is a nucleic acid sequence having at least 75% (e.g., at least about 79%, about 80%, about 85%, about 90%, about 95%, about 99% or about 100%) homology or complementarity to any nucleotide sequence selected from SEQ ID NOs: 280-416.

A small activating nucleic acid molecule of the present invention comprises a double-stranded small activating nucleic acid molecule targeting the promoter region of the ATOH1 gene comprising a first nucleic acid strand and a second nucleic acid strand, wherein the first nucleic acid strand has at least 75% (e.g., at least about 79%, about 80%, about 85%, about 90%, about 95%, about 99% or about 100%) homology or complementarity to any continuous sequence of 16 to 35 nucleotides in length in positions −578 to −386 (hotspot 1, SEQ ID NO: 2), positions −339 to −291 (hotspot 2, SEQ ID NO: 3), positions −162 to −86 (hotspot 3, SEQ ID NO: 4) or positions −55 to −34 (hotspot 4, SEQ ID NO: 5) away from the transcription start site of the ATOH1 gene promoter, and the first nucleic acid strand and the second nucleic acid strand can complementarily form a double-stranded nucleic acid structure capable of activating the expression of ATOH1 in a cell.

The first nucleic acid strand and the second nucleic acid strand of a small activating nucleic acid molecule of the present invention can be present on either two different nucleic acid strands or on the same nucleic acid strand. When the first nucleic acid strand and the second nucleic acid strand are located on two different strands, at least one strand of a small activating nucleic acid molecule can have overhangs at the 5′ terminus and/or the 3′ terminus, e.g. overhangs of 0 to 6 nucleotides in length at 3′ terminus, such that the overhangs of 0, 1, 2, 3, 4, 5 or 6 nucleotides in length. Preferably, both strands of a small activating nucleic acid molecule of the present invention have overhangs; more preferably, the 3′ terminus of both strands of a small activating nucleic acid molecule can have overhangs of 0 to 6 nucleotides in length, e.g., overhangs of 0, 1, 2, 3, 4, 5 or 6 nucleotides in length; most preferably overhangs of 2 or 3 nucleotides in length. Preferably, the nucleotide of the overhang is dT.

A small activating nucleic acid molecule of the present invention can also comprise a small activating nucleic acid molecule capable of forming a double-stranded region hairpin structure, e.g., a single-stranded small activating RNA molecule. In one embodiment, a small activating nucleic acid molecule of the present invention comprises a single-stranded small activating RNA molecule targeting the promoter region of the ATOH1 gene, wherein the single-stranded small activating nucleic acid molecule can form a double-stranded region hairpin structure. Preferably, when the first nucleic acid strand and the second nucleic acid strand are present on the same nucleic acid strand, a small activating nucleic acid molecule of the present invention can be a hairpin single-stranded nucleic acid molecule, wherein the first nucleic acid strand and the second nucleic acid strand have complementary regions capable of forming a double-stranded nucleic acid structure, and the double-stranded nucleic acid structure can promote the expression of ATOH1 in a cell with, for example, a RNA activation mechanism.

In the aforementioned small activating nucleic acid molecule, the first nucleic acid strand and the second nucleic acid strand can have 16 to 35 nucleotides (e.g., 16, 17, 18, 19, 20, 21, 22, 23, 24, 25, 26, 27, 28, 29, 30, 31, 32, 33, 34 or 35 nucleotides) in length.

In one embodiment, the first nucleic acid strand of a small activating nucleic acid molecule of the present invention has at least 75% (e.g., at least about 79%, about 80%, about 85%, about 90%, about 95%, about 99% or about 100%) identity or homology to any nucleotide sequence selected from SEQ ID NOs: 6-142, and the second nucleic acid strand of a small activating nucleic acid molecule has at least 75% (e.g., at least about 79%, about 80%, about 85%, about 90%, about 95%, about 99% or about 100%) identity or homology to any nucleotide sequence selected from SEQ ID NOs: 143-279. In one embodiment, the first nucleic acid strand of a small activating nucleic acid molecule of the present invention comprises a nucleic acid sequence having at least 75% (e.g., at least about 79%, about 80%, about 85%, about 90%, about 95%, about 99% or about 100%) identity or homology to any nucleotide sequence selected from SEQ ID NOs: 6-142, or consists of a nucleic acid sequence having at least 75% (e.g., at least about 79%, about 80%, about 85%, about 90%, about 95%, about 99% or about 100%) identity or homology to any nucleotide sequence selected from SEQ ID NOs: 6-142; the second nucleic acid strand of a small activating nucleic acid molecule of the present invention comprises a nucleic acid sequence having at least 75% (e.g., at least about 79%, about 80%, about 85%, about 90%, about 95%, about 99% or about 100%) identity or homology to any nucleotide sequence selected from SEQ ID NOs: 143-279, or consists of a nucleic acid sequence having at least 75% (e.g., at least about 79%, about 80%, about 85%, about 90%, about 95%, about 99% or about 100%) identity or homology to any nucleotide sequence selected from SEQ ID NOs: 143-279. In a specific embodiment, the first nucleic acid strand of a small activating nucleic acid molecule of the present invention can comprise or be selected from any nucleotide sequence set forth in SEQ ID NOs: 6-142, and the second strand can comprise or be selected from any nucleotide sequence set forth in SEQ ID NOs: 143-279. In one embodiment, a small activating nucleic acid molecule described herein can be synthesized, transcribed in vitro or expressed by a vector.

All the nucleotides in a small activating nucleic acid molecule described herein can be natural non-chemically modified nucleotides or can comprise at least one modification. In one embodiment, the modification in a small activating nucleic acid molecule described herein can comprise chemical modification, for example, at least one nucleotide can have chemical modification, and the chemical modification used in the present invention can comprise or be selected from one or more or any combination of the following modifications:

(1) a modification of a phosphodiester bond of nucleotides in the nucleotide sequence of a small activating nucleic acid molecule;

(2) a modification of 2′-OH of a ribose in the nucleotide sequence of a small activating nucleic acid molecule;

(3) a modification of a base in the nucleotide sequence of a small activating nucleic acid molecule; and

(4) at least one nucleotide in the nucleotide sequence of a small activating nucleic acid molecule being a locked nucleic acid.

The chemical modifications described herein are well-known to those skilled in the art, and the modification of the phosphodiester bond refers to the modification of oxygen in the phosphodiester bond, including, but not limited to, phosphorothioate modification and boranophosphate modification. Both modifications can stabilize an saRNA structure and maintain high specificity and high affinity for base pairing.

The ribose modification refers to the modification of 2′-OH in pentose of a nucleotide, i.e., the introduction of some substituents into hydroxyl positions of the ribose, for example, including but not limited to 2′-fluoro modification, 2′-oxymethyl modification, 2′-oxyethylidene methoxy modification, 2,4′-dinitrophenol modification, locked nucleic acid (LNA), 2′-amino modification, 2′-deoxy modification, and the like.

The base modification refers to the modification of the base of a nucleotide, for example, including but not limited to 5′-bromouracil modification, 5′-iodouracil modification, N-methyluracil modification, 2,6-diaminopurine modification, and the like.

These modifications can increase the bioavailability of a small activating nucleic acid molecule, improve affinity to a target sequence and enhance resistance to nuclease hydrolysis in a cell.

In addition, in order to promote the access of a small activating nucleic acid molecule into a cell, on the basis of the aforementioned modifications, a lipophilic group, such as cholesterol, can be introduced on the terminus of the first nucleic acid strand and/or the second nucleic acid strand of a small activating nucleic acid molecule to facilitate the interaction with the promoter region of a gene in the cell nucleus, as the cell membrane and nuclear membrane are composed of lipid bilayers.

After contacting and entering a cell, a small activating nucleic acid molecule disclosed herein can effectively activate or up-regulate the expression of ATOH1 gene in a cell, preferably up-regulate the expression by at least 10%.

Another aspect of the present invention also relates to a nucleic acid encoding a small activating nucleic acid molecule of the invention. In one embodiment, the nucleic acid is a DNA molecule.

Another aspect of the present invention provides a cell comprising the aforementioned small activating nucleic acid molecule or the nucleic acid encoding a small activating nucleic acid molecule described herein. In one embodiment, a small activating nucleic acid molecule of the present invention can be a double-stranded small activating nucleic acid molecule targeting the promoter region of the ATOH1 gene, which comprises a first nucleic acid strand and a second nucleic acid strand. In another embodiment, a small activating nucleic acid molecule of the present invention can be a single-stranded small activating nucleic acid molecule targeting the promoter region of ATOH1 gene.

Another aspect of the present invention provides a composition (e.g., a pharmaceutical composition). The composition comprises a small activating nucleic acid molecule of the present invention or a nucleic acid encoding a small activating nucleic acid molecule described herein and, optionally, a pharmaceutically acceptable carrier. In one embodiment, the pharmaceutically acceptable carrier can comprise or be selected from a liposome, a high-molecular polymer, and a polypeptide.

In another aspect of the present invention, a formulation is provided which comprises a small activating nucleic acid molecule described in the present invention, a nucleic acid encoding a small activating nucleic acid molecule described in the present invention, or a cell comprising a small activating nucleic acid molecule of the present invention or a nucleic acid encoding a small activating nucleic acid molecule of the present invention, or a composition comprising a small activating nucleic acid molecule of the present invention.

In another aspect of the present invention, a kit is provided, which comprises a small activating nucleic acid molecule of the present invention, a nucleic acid encoding a small activating nucleic acid molecule described herein, or a cell comprising a small activating nucleic acid molecule of the present invention or a nucleic acid encoding a small activating nucleic acid molecule of the present invention, or a composition comprising a small activating nucleic acid molecule of the present invention.

Another aspect of the present invention relates to uses of a small activating nucleic acid molecule of the present invention, the nucleic acid encoding a small activating nucleic acid molecule of the present invention, a cell comprising a small activating nucleic acid molecule of the present invention, or the nucleic acid encoding a small activating nucleic acid molecule of the present invention, or a composition comprising a small activating nucleic acid molecule of the present invention in the preparation of a drug or formulation for activating or upregulating the expression of ATOH1 in a cell.

Another aspect of the present invention also relates to a method for activating or upregulating the expression of ATOH1 in a cell. The method comprises administering a small activating nucleic acid molecule of the present invention, the nucleic acid encoding a small activating nucleic acid molecule of the present invention, or the composition or formulation comprising a small activating nucleic acid molecule of the present invention to the cell. In one embodiment, the method for activating/up-regulating the expression of ATOH1 gene in a cell comprises administering a small activating nucleic acid molecule described in the present invention, the nucleic acid encoding a small activating nucleic acid molecule of the present invention, or the composition or formulation comprising a small activating nucleic acid molecule of the present invention to the cell wherein the cell includes, but not limited to, a cancer cell, a neural cell, e.g., a spinal cord cell, an inner ear hair cell, an inner ear support cell, a cerebellar neuron, a proprioceptive system neuron, a non-neural cell, and the like. In one embodiment, the cancer cell can be a cell from a solid tumor, e.g., liver cancer, lung cancer, bladder cancer, prostate cancer, and glioma. The aforementioned cell can be in vitro, e.g., a cell line or a cell strain, or can be present in a mammalian body, e.g., a human body.

A small activating nucleic acid molecule of the present invention can be directly introduced into a cell, or can be produced in the cell after a nucleotide sequence encoding a small activating nucleic acid molecule of the present invention is introduced into the cell. The cell is preferably a mammalian cell, more preferably a human cell. The aforementioned cell can be in vitro, such as a cell line or a cell strain, or can be present in a mammalian body, e.g., a human body. The human body can be a patient suffering from a disease or condition related to insufficient or decreased expression of the ATOH1 protein. A small activating nucleic acid molecule of the present invention can be administered at a sufficient dose to treat the disease or condition related to a deficiency of the ATOH1 protein or insufficient or decreased expression of ATOH1 protein. Specifically, the disease or condition related to deficiency of ATOH1 protein amount or insufficient or decreased expression of ATOH1 protein can comprise, for example, tumors, e.g., solid tumors, and the solid tumors can comprise, for example, liver cancer, lung cancer, bladder cancer, prostatic cancer, glioma, and the like.

Another aspect of the present invention provides an isolated small activating nucleic acid molecule acting site of the ATOH1 gene, which has any continuous sequence of 16 to 35 nucleotides in length in the promoter region of ATOH1 gene, and preferably, the acting site comprises or is selected from any continuous sequence of 16 to 35 nucleotides in length in any of the sequences set forth in SEQ ID NOs: 2-5. Specifically, the acting site can comprise or be selected from any nucleotide sequence set forth in SEQ ID NOs: 280-416.

Another aspect of the present invention relates to a method for treating a disease or condition related to insufficient or decreased expression of the ATOH1 protein in a subject, which comprises administering a small activating nucleic acid molecule of the present invention, a nucleic acid encoding a small activating nucleic acid molecule of the present invention, a cell comprising a small activating nucleic acid molecule of the present invention or a nucleic acid encoding a small activating nucleic acid molecule of the present invention or the composition comprising a small activating nucleic acid molecule of the present invention at a curative dose to the subject. In one embodiment, a method for treating a disease or condition related to insufficient or decreased expression of the ATOH1 protein in a subject in the present invention comprises administering a small activating nucleic acid molecule of the present invention, the nucleic acid encoding a small activating nucleic acid molecule of the present invention, the cell comprising a small activating nucleic acid molecule of the present invention or a nucleic acid encoding a small activating nucleic acid molecule of the present invention or a composition comprising a small activating nucleic acid molecule of the present invention, and a low-molecular-weight compound, antibody, polypeptide, or protein at a curative dose to the subject. The subject can be a mammal, e.g., as a human. In one embodiment, a disease or condition related to insufficient or decreased expression of the ATOH1 protein can comprise, e.g., hearing loss and tumor diseases, e.g., colorectal cancer.

Another aspect of the present invention relates to a method for treating hearing loss in a subject, which comprises administering a small activating nucleic acid molecule of the present invention, a nucleic acid encoding a small activating nucleic acid molecule of the present invention, a cell comprising a small activating nucleic acid molecule of the present invention, or a nucleic acid encoding a small activating nucleic acid molecule of the present invention, or a composition or formulation comprising a small activating nucleic acid molecule of the present invention at a curative dose to the subject. In one embodiment, the method for treating hearing loss in a subject comprises administering a small activating nucleic acid molecule of the present invention, the nucleic acid encoding a small activating nucleic acid molecule of the present invention, the cell comprising a small activating nucleic acid molecule of the present invention, or a nucleic acid encoding a small activating nucleic acid molecule of the present invention, or the composition comprising a small activating nucleic acid molecule of the present invention, and a low-molecular-weight compound, antibody, polypeptide or protein at a curative dose to the subject. The subject can be a mammal, e.g., a human. Hearing loss or hearing impairment refers to a partial or complete loss of hearing with less sensitivity to daily speech. In 1997, the World Health Organization (WHO) issued standards that divided the severity of hearing loss into 4 levels according to average hearing loss at four frequency points of 500 Hz, 1000 Hz, 2000 Hz and 4000 Hz: 26-40 dB is mild hearing loss, 41-60 dB is moderate hearing loss, 61-80 dB is severe hearing loss, and greater than 80 dB is extremely severe hearing loss.

Another aspect of the present invention relates to uses of a small activating nucleic acid molecule of the present invention, a nucleic acid encoding a small activating nucleic acid molecule of the present invention, a cell comprising a small activating nucleic acid molecule of the present invention or a nucleic acid encoding a small activating nucleic acid molecule of the present invention or a composition comprising a small activating nucleic acid molecule of the present invention in the preparation of a drug for treating a disease or condition related to insufficient or decreased expression of the ATOH1 protein. The subject can be a mammal, e.g., a human. In one embodiment, the disease related to insufficient or decreased expression of the ATOH1 protein can comprise, e.g.,, hearing loss and tumor diseases, e.g., colorectal cancer.

In one embodiment, uses of a small activating nucleic acid molecule described in the present invention, a nucleic acid encoding a small activating nucleic acid molecule of the present invention, a cell comprising a small activating nucleic acid molecule of the present invention or a nucleic acid encoding a small activating nucleic acid molecule of the present invention, or a composition comprising a small activating nucleic acid molecule of the present invention in the preparation of a drug for hearing loss.

Another aspect of the present invention relates to uses of a small activating nucleic acid molecule of the present invention, a nucleic acid encoding a small activating nucleic acid molecule of the present invention, a cell comprising a small activating nucleic acid molecule of the present invention or a nucleic acid encoding a small activating nucleic acid molecule of the present invention, or a composition or formulation comprising a small activating nucleic acid molecule of the present invention in the preparation of a drug or pharmaceutical composition for treating a disease or condition related to insufficient or decreased expression of ATOH1 protein.

In one embodiment, uses of a small activating nucleic acid molecule described in the present invention, a nucleic acid encoding a small activating nucleic acid molecule of the present invention, a cell comprising a small activating nucleic acid molecule of the present invention or a nucleic acid encoding a small activating nucleic acid molecule of the present invention, or a composition or formulation comprising a small activating nucleic acid molecule of the present invention in the preparation of a drug or pharmaceutical composition for treating hearing loss.

### ADVANTAGES OF THE PRESENT INVENTION

A small activating nucleic acid molecule, such as a small activating RNA (saRNA) is capable of activating or upregulating the expression of the ATOH1 gene provided by the present invention and can persistently activate the ATOH1 gene, thereby efficiently and specifically upregulating or restoring the expression of the ATOH1 gene and protein while featuring lower toxic and side effects, which can be used in treating a disease or symptom related to insufficient or decreased expression of the ATOH1 protein such as hearing loss, and in preparing a drug or formulation for treating the disease or symptom related to insufficient or decreased expression of the ATOH1 protein.

### BRIEF DESCRIPTION OF THE DRAWINGS

FIG. 1 shows changes in the expression of ATOH1 mRNA mediated by ATOH1 saRNA. HEK293T cells were transfected by 403 saRNAs targeting the ATOH1 promoter and mRNA expression of ATOH1 was analyzed 72 h later using QuantiGene 2.0. Shown in the drawing are the changes in ATOH1 mRNA expression relative to control treatment (mock) ranked from −637 to −19 according to the saRNA target positions in the ATOH1 promoter.

FIG. 2 shows the hotspot region of functional saRNA in the ATOH1 promoter. HEK293T cells were transfected by 403 saRNAs targeting ATOH1 promoter and mRNA expression of ATOH1 was analyzed 72 h later using QuantiGene 2.0. Shown in the drawing are changes in ATOH1 mRNA expression relative to control treatment (mock) ranked from −637 to -19 according to the target positions of the saRNA in the ATOH1 promoter. The dotted line in FIG. 2 is the limit of 0.5 (log₂) fold increase. The dotted frames show hotspot regions (H1-H4) for functional saRNAs.

FIG. 3 shows the screening results for two-step RT-PCR validated by QuantiGene in HEK293T cells. HEK293T cells were transfected with saRNA at a final concentration of 10 nM for 72 h. RNA was extracted using an Qiagen Rneasy kit and qPCR amplification was performed with a 7500 FAST real-time PCR system after reverse transcription. An HPRT1 gene was amplified as internal control. The relative mRNA expression values of ATOH1 after treating cells with a single saRNA are shown. Mock, dsCon2, and RAG9-871i are represented as blank transfection, sequence-independent double-stranded RNA transfection, and small interference RNA control transfection, respectively.

FIG. 4 shows the screening results for two-step RT-PCR validated by Quantigene in NKEK cells. Normal human epidermal keratinocytes (NHEK) cells were transfected with saRNA at a final concentration of 10 nM for 72 h. RNA was extracted using a Qiagen Rneasy kit and qPCR amplification was performed with a 7500 FAST real-time PCR system after reverse transcription. An HPRT1 gene was amplified as internal control. The relative mRNA expression values of ATOH1 after treatment of cells with a single saRNA are shown. Mock, dsCon2, and RAGS-871i are represented as blank transfection, sequence-independent double-stranded RNA transfection, and small interference RNA control transfection, respectively.

FIG. 5 shows ATOH1 saRNA activating the expression of ATOH1 protein. HEK293T cells were transfected with saRNA at a final concentration of 10 nM for 72 h. After collecting the cells and extracting total cell protein, ATOH1 protein expression was detected by Western Blot and α/β-tubulin was used as an internal control. The relative ATOH1 protein expression values after cell treatment with a single saRNA are shown. Mock, dsCon2, and RAGS-871i are represented as blank transfection, sequence-independent double-stranded RNA transfection, and small interference RNA control transfection, respectively.

### DETAILED DESCRIPTION

In the present invention, the related terms are defined as follows.

The term “complementarity” as used herein refers to the capability of forming base pairs between two oligonucleotide strands. The base pairs are generally formed through hydrogen bonds between nucleotides in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing).

Complementarity comprises complete complementarity and incomplete complementarity. “Complete complementarity” or “100% complementarity” means that each nucleotide from the first oligonucleotide strand can form a hydrogen bond with a nucleotide at a corresponding position in the second oligonucleotide strand in the double-stranded region of the double-stranded oligonucleotide molecule without “mispairing”. “Incomplete complementarity” means that not all the nucleotide units of the two strands are bonded with each other by hydrogen bonds. For example, for two oligonucleotide strands each of 20 nucleotides in length in the double-stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. Substantial complementarity refers to at least about 75%, about 79%, about 80%, about 85%, about 90%, about 95%, about 99% or about 100% complementarity.

The term “oligonucleotide” as used herein refers to polymers of nucleotides, and includes, but is not limited to, single-stranded or double-stranded molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides. The oligonucleotide for activating target gene transcription described herein is a small activating nucleic acid molecule.

The terms “oligonucleotide strand” and “oligonucleotide sequence” as used herein can be used interchangeably, referring to a generic term for short nucleotide sequences having less than 35 bases (including nucleotides in deoxyribonucleic acid (DNA) or ribonucleic acid (RNA)). In the present invention, an oligonucleotide strand can have any of 16 to 35 nucleotides in length.

As used herein, the term “first nucleic acid strand” can be a sense strand or an antisense strand. The sense strand of a small activating RNA refers to a nucleic acid strand contained in a small activating RNA duplex which has identity to the coding strand of the promoter DNA sequence of a target gene, and the antisense strand refers to a nucleic acid strand in a small activating RNA duplex which is complementary to the sense strand.

As used herein, the term “second nucleic acid strand” can also be a sense strand or an antisense strand. If the first oligonucleotide strand is a sense strand, the second oligonucleotide strand is an antisense strand; if the first oligonucleotide strand is an antisense strand, the second oligonucleotide strand is a sense strand.

The term “gene” as used herein refers to all nucleotide sequences required to encode a polypeptide chain or to transcribe a functional RNA. “Gene” can be an endogenous or fully or partially recombinant gene for a host cell (for example, because an exogenous oligonucleotide and a coding sequence for encoding a promoter are introduced into a host cell, or a heterogeneous promoter adjacent to an endogenous encoding sequence is introduced into a host cell). For example, the term “gene” comprises a nucleic acid sequence consisting of exons and introns. Protein-encoding sequences are, for example, sequences contained within exons in an open reading frame between an initiation codon and a termination codon, and as used herein, “gene” can comprise such as a gene regulatory sequence, such as a promoter, an enhancer, and all other sequences known in the art for controlling the transcription, expression or activity of another gene, no matter whether the gene comprises a coding sequence or a non-coding sequence. In one case, for example, “gene” can be used to describe a functional nucleic acid comprising a regulatory sequence such as a promoter or an enhancer. The expression of a recombinant gene can be controlled by one or more types of heterogeneous regulatory sequences.

The term “target gene” as used herein can refer to nucleic acid sequences naturally present in organisms, transgenes, viral or bacterial sequences, can be chromosomes or extrachromosomal genes, and/or can be transiently or stably transfected or incorporated into cells and/or chromatins thereof. The target gene can be a protein-encoding gene or a non-protein-encoding gene (such as a microRNA gene and a long non-coding RNA gene). The target gene generally contains a promoter sequence, and the positive regulation for the target gene can be achieved by designing a small activating nucleic acid molecule having sequence identity (also called homology) to the promoter sequence, characterized as the up-regulation of expression of the target gene. “Sequence of a target gene promoter” refers to a non-coding sequence of the target gene, and the reference of the sequence of a target gene promoter in the phrase “complementary to the sequence of a target gene promoter” of the present invention refers to a coding strand of the sequence, also known as a non-template strand, i.e., a nucleic acid sequence having the same sequence as the coding sequence of the gene. “Target” or “target sequence” refers to a sequence fragment in the sequence of a target gene promoter which is homologous or complementary with a sense oligonucleotide strand or an antisense oligonucleotide strand of a small activating nucleic acid molecule.

As used herein, the terms “sense strand” and “sense nucleic acid strand” can be used interchangeably, and the sense oligonucleotide strand of a small activating nucleic acid molecule refers to the first nucleic acid strand having identity to the coding strand of the sequence of a target gene promoter in a small activating nucleic acid molecule duplex.

As used herein, the terms “antisense strand” and “antisense nucleic acid strand” can be used interchangeably, and the antisense oligonucleotide strand of a small activating nucleic acid molecule refers to the second nucleic acid strand complementary with the sense oligonucleotide strand in a small activating nucleic acid molecule duplex.

The term “coding strand” as used herein refers to a DNA strand in the target gene which cannot be used for transcription, and the nucleotide sequence of this strand is the same as that of an RNA produced from transcription (in the RNA, T in DNA is replaced by U). The coding strand of the double-stranded DNA sequence of the target gene promoter described herein refers to a promoter sequence on the same DNA strand as the DNA coding strand of the target gene.

The term “template strand” as used herein refers to the other strand complementary with the coding strand in the double-stranded DNA of the target gene, i.e., the strand that, as a template, can be transcribed into RNA, and this strand is complementary with the transcribed RNA (A to U and G to C). In the process of transcription, RNA polymerase binds to the template strand, moves along the 3′→5′ direction of the template strand, and catalyzes the synthesis of the RNA along the 5′→3′ direction. The template strand of the double-stranded DNA sequence of the target gene promoter described herein refers to a promoter sequence on the same DNA strand as the DNA template strand of the target gene.

The term “promoter” as used herein refers to a sequence which plays a regulatory role for the transcription of a protein-coding or RNA-coding nucleic acid sequence by associating with them spatially. Generally, a eukaryotic gene promoter contains 100 to 5000 base pairs, although this length range is not intended to limit the term “promoter” as used herein. Although the promoter sequence is generally located at the 5′ terminus of a protein-coding or RNA-coding sequence, the promoter sequence can also exist in exon and intron sequences.

The term “transcription start site” as used herein refers to a nucleotide marking the transcription start on the template strand of a gene. The transcription start site can appear on the template strand of the promoter region. A gene can have more than one transcription start site.

The term “identity” or “homology” as used herein means that one oligonucleotide strand (a sense or an antisense strand) of a small activating RNA has similarity to a coding strand or a template strand in a region of the promoter sequence of a target gene. As used herein, the “identity” or “homology” can be at least about 75%, about 79%, about 80%, about 85%, about 90%, about 95%, about 99% or about 100%.

The term “overhang” as used herein refers to non-base-paired nucleotides at the terminus (5′ or 3′) of an oligonucleotide strand, which is formed by one strand extending out of the other strand in a double-stranded oligonucleotide. A single-stranded region extending out of the 3′ terminus and/or 5′ terminus of a duplex is referred to as an overhang.

As used herein, the terms “gene activation”, “activating gene expression”, “gene up-regulation” and “up-regulating gene expression” can be used interchangeably, and mean an increase in transcription, translation, expression or activity of a certain nucleic acid as determined by measuring the transcriptional level, mRNA level, protein level, enzymatic activity, methylation state, chromatin state or configuration, translation level or the activity or state in a cell or biological system of a gene. These activities or states can be determined directly or indirectly. In addition, “gene activation”, “activating gene expression”, “gene up-regulation” or “up-regulating gene expression” refers to an increase in activity associated with a nucleic acid sequence, regardless of the mechanism of such activation. For example, the nucleic acid sequence plays a regulatory role as a regulatory sequence, the nucleic acid sequence is transcribed into RNA and the RNA is translated into a protein, thereby increasing the expression of the protein.

As used herein, the terms “small activating RNA”, “saRNA”, and “small activating nucleic acid molecule” can be used interchangeably, and refer to a nucleic acid molecule that can up-regulate target gene expression and can be composed of the first nucleic acid fragment (antisense nucleic acid strand, also referred to as antisense oligonucleotide strand) containing a nucleotide sequence having sequence identity or homology with the non-coding nucleic acid sequence (e.g., a promoter and an enhancer) of a target gene and the second nucleic acid fragment (sense nucleic acid strand, also referred to as sense oligonucleotide strand) containing a nucleotide sequence complementary with the first nucleic acid fragment, wherein the first nucleic acid fragment and the second nucleic acid fragment form a duplex. A small activating nucleic acid molecule can also be composed of a synthesized or vector-expressed single-stranded RNA molecule that can form a hairpin structure by two complementary regions within the molecule, wherein the first region comprises a nucleotide sequence having sequence identity to the target sequence of a promoter of a gene, and the second region comprises a nucleotide sequence which is complementary to the first region. The length of the duplex region of a small activating nucleic acid molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45, or about 50 base pairs. In addition, the terms “saRNA”, “small activating RNA”, and “small activating nucleic acid molecule” also comprise nucleic acids other than the ribonucleotide, including, but not limited to, modified nucleotides or analogues.

As used herein, the term “hotspot” refers to a gene promoter region of at least 30 bp in length. The gathering of targets of functional small activating nucleic acid molecules appears in these hotspot regions, wherein at least 40% of a small activating nucleic acid molecules targeting these hotspot regions can induce a 1.1-fold or more change in the mRNA expression of a target gene.

As used herein, the term “synthesis” refers to a method for synthesis of an oligonucleotide, including any method allowing RNA synthesis, such as chemical synthesis, in vitro transcription, and/or vector-based expression.

According to the present invention, the expression of the ATOH1 gene is up-regulated by RNA activation, and a related disease (particularly hearing loss) is treated by increasing the expression of the full-length ATOH1 protein. The ATOH1 gene in the present invention is sometimes also called a target gene.

The method for preparing a small activating nucleic acid molecule provided by the present invention comprises sequence design and sequence synthesis.

The synthesis of the sequence of a small activating nucleic acid molecule of the present invention can be performed by adopting a chemical synthesis or can be entrusted to a biotechnology company specialized in nucleic acid synthesis.

Generally speaking, the chemical synthesis comprises the following four steps: (1) synthesis of oligomeric ribonucleotides, (2) deprotection, (3) purification and isolation; and (4) desalination and annealing.

For example, the specific steps for chemically synthesizing the saRNA described herein are as follows:

### (1) Synthesis of Oligomeric Ribonucleotides

Synthesis of 1 μM RNA was set in an automatic DNA/RNA synthesizer (e.g., Applied Biosystems EXPEDITE8909), and the coupling time of each cycle was also set as 10 to 15 min. With a solid phase-bonded 5′-O-p-dimethoxytriphenylmethyl-thymidine substrate as an initiator, one base was bonded to the solid phase substrate in the first cycle, and then, in the n^{th} (19≥n≥2) cycle, one base was bonded to the base bonded in the n-1^{th} cycle. This process was repeated until the synthesis of the whole nucleic acid sequence was completed.

### (2) Deprotection

The solid phase substrate bonded with the saRNA was put into a test tube, and 1 mL of a mixed solution of ethanol and ammonium hydroxide (volume ratio: 1:3) was added to the test tube. The test tube was then sealed and placed in an incubator, and the mixture was incubated at 25-70° C. for 2 to 30 h. The solution containing the solid phase substrate bonded with the saRNA was filtered, and the filtrate was collected. The solid phase substrate was rinsed with double distilled water twice (1 mL each time), and the filtrate was collected. The collected eluent was combined and dried under vacuum for 1 to 12 h. Then the solution was added with 1 mL of a solution of tetrabutylammonium fluoride in tetrahydrofuran (1 M), let stand at room temperature for 4 to 12 h, followed by addition of 2 mL of n-butanol. Precipitate was collected to give a single-stranded crude product of saRNA by high-speed centrifugation.

### (3) Purification and Isolation

The resulting crude product of saRNA was dissolved in 2 mL of triethylamine acetate solution with a concentration of 1 mol/L, and the solution was separated by a reversed-phase C18 column of high pressure liquid chromatography to give a purified single-stranded product of saRNA.

### (4) Desalination and Annealing

Salts were removed by gel filtration (size exclusion chromatography). A single sense oligomeric ribonucleic acid strand and a single antisense oligomeric ribonucleic acid strand were mixed in a 1 to 2 mL of buffer (10 mM Tris, pH=7.5-8.0, 50 mM NaCl) at a molar ratio of 1:1. The solution was heated to 95° C., and was then slowly cooled to room temperature to give a solution containing saRNA.

It was discovered in this study that after being introduced into a cell, the aforementioned saRNA could effectively increase the mRNA and protein expression of the full-length ATOH1.

The present invention will be further illustrated with reference to specific examples and drawings below. It should be understood that these examples are merely intended to illustrate the present invention rather than limit the scope of the present invention. In the following examples, study methods without specific conditions were generally in accordance with conventional conditions, such as conditions described in Sambrook, et al., Molecular Cloning: Laboratory Manual (New York: Cold Spring Harbor Laboratory Press, 1989), or conditions recommended by the manufacturer.

### EXAMPLES

### Example 1: Design and Synthesis of Small Activating Nucleic Acid Molecule Targeting ATOH1 Promoter

To screen functional saRNAs capable of activating ATOH1 gene expression, a ATOH1 promoter sequence with a length of 637 bps (−637 bp to −1 bp; SEQ ID NO:1) was used as a template and an saRNA target with a lengths of 19 nucleotides were obtained by moving a base each time. Target sequences were filtered and the criteria for retaining target sequences were: (1) GC content between 30% and 80%, (2) with less than 5 continuous identical nucleotides, (3) with 3 or less dinucleotide repeat sequences; and (4) with 3 or less trinucleotide repeat sequences. There were 402 target sequences remaining after filtration and these entered the screening process as candidates. In addition, one target at the position −1407 bp of the ATOH1 promoter was selected as a candidate target sequence, and the 403 target sequences were selected as candidates to enter screening process.

Each sense strand and each antisense strand in the double-stranded small activating RNAs used in the study were 21 nucleotides in length. The 19 nucleotides in the 5′ region of the first nucleic acid strand, e.g., the sense strand of the double-stranded saRNA, had 100% identity to the target sequence of the promoter, and the 3′ terminus of the first nucleic acid strand contained a TT sequence. The 19 nucleotides in the 5′ region of the second nucleic acid strand were fully complementary with the first ribonucleic acid strand sequence, and the 3′ terminus of the second nucleic acid strand contained a TT sequence. The aforementioned two strands of the double-stranded saRNA were mixed at a molar ratio of 1:1, and after annealing, and a double-stranded saRNA was formed. The sequence of the ATOH1 promoter corresponds to position 1 to position 637 from 5′ to 3′ of SEQ ID No: 1 in the sequence listing:

| | |
|---|---|
| −637 | aaatgcttgg gcatcagaag tgggagctgt gatcctagct |
| | tgggggcagc |
| −587 | acagggtagg cggccttctc tctgctttga gtggcttctg |
| | ggcgcctggc |
| −537 | gggtccagaa tcgcccagag ccgcccgcgg tcgtgcacat |
| | ctgacccgag |
| −487 | tcagcttggg caccagccga gagccggctc cgcaccgctc |
| | ccgcacccca |
| −437 | gccgccgggg tggtgacaca caccggagtc gaattacagc |
| | cctgcaatta |
| −387 | acatatgaat ctgacgaatt taaaagaagg aaaaaaaaaa |
| | aaaaacctga |
| −337 | gcaggcttgg gagtcctctg cacacaagaa cttttctcgg |
| | ggtgtaaaaa |
| −287 | ctctttgatt ggctgctcgc acgcgcctgc ccgcgccctc |
| | cattggctga |
| −237 | gaagacacgc gaccggcgcg aggagggggt tgggagagga |
| | gcggggggag |
| −187 | actgagtggc gcgtgccgct ttttaaaggg gcgcagcgcc |
| | ttcagcaacc |
| −137 | ggagaagcat agttgcacgc gacctggtgt gtgatctccg |
| | agtgggtggg |
| −87 | ggagggtcga ggagggaaaa aaaaataaga cgttgcagaa |
| | gagacccgga |
| −37 | aagggccttt tttttggttg agctggtgtc ccagtgc |

### Example 2: High-throughput Screening of saRNAs Targeting ATOH1 Promoter

### (1) Cell Culture and Transfection

Normal Human Epidermal Keratinocytes (NHEK) and human renal embryonic cells (HEK293T) were cultured in DMEM media (Gibco). All the media contained 10% bovine calf serum (Sigma-Aldrich) and 1% penicillin/streptomycin (Gibco). The cells were cultured at 37 ° C. under 5% CO2. According to the instructions provided by the manufacturer, RNAiMax (Invitrogen, Carlsbad, Calif.) was used to transfect small activating RNAs at a concentration of 10 nM (unless otherwise specified).

### (2) QuantiGene 2.0 Assay

Cells were plated into a 96-well plate at 4000 cells/well and transfected with oligonucleotide duplexes, and 72 h after transfection, the mRNA levels of target genes were quantitatively determined with a QuantiGene 2.0 kit (AffyMetrix, product numbers were QGS-1000, ATOH1 Assay ID: SA-6000952, and HPRT Assay ID: SA-10030). QuantiGene 2.0 assay is based on hybridization technology, wherein mRNA levels were directly quantified with gene-specific probes. The experimental procedure was briefly described as follows: a lysis solution was added to lyse the transfected cells, and the cell lysates were added into a capture well plate coated with probe for ATOH1 and HPRT1 (housekeeping gene) for hybridizing overnight at 55° C. In order to enhance hybridization signal, hybridizations with 2.0 PreAMP Probe, 2.0 AMP Probe and 2.0 Label Probe (provided by Quantigene 2.0 kit) were conducted sequentially in 100 μL of a corresponding buffer solution (provided by Quantigene 2.0 kit). All the hybridizations were conducted with shaking at 50 to 55° C. for 1 h. After the last wash step, 2.0 Substrate was added to the solution and incubated at room temperature for 5 min. Optical signals were detected with an Infinite 200 PRO plate reader (Tecan, Switzerland).

### (3) Screening of Functional saRNAs

In order to obtain functional saRNAs capable of activating transcription of ATOH1, HEK293T cells were transfected with the above 403 saRNAs at a concentration of 10 nM, and light signals were analyzed 72 h later by the method described in QuantiGene. As shown in Table 1, 32 (7.9%) and 105 (26.1%) saRNAs showed high and mild activating activities, and 266 (66.0%) saRNAs had no influence on the expression of ATOH1. The maximum activation was 5.88 fold. These saRNAs with activating activity are referred to as functional saRNA.

TABLE 1
High-throughput screening results of ATOH1

| | log₂ value (fold) | Number of | |
|---|---|---|---|
| saRNA activity | of changes in ATOH1 | saRNAs | Percentage |
| High activation | ≥1.00 (2.00)~≤2.557 (5.883) | 32 | 7.9% |
| Mild activation | ≥0.14 (1.10)~<1.00 (2.00) | 105 | 26.1% |
| No activating | <0.14 (1.10) | 266 | 66.0% |
| effect | | | |
| Sum | | 403 | 100% |

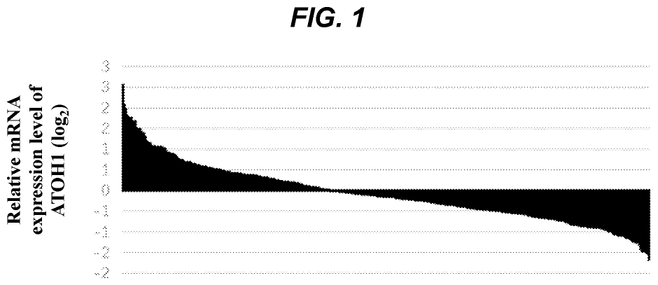

FIG. 1 further shows the activity distribution of the ATOH1 saRNAs from high activation to high inhibition.

TABLE 2
Active saRNA sequences, active target sequences thereof and resulting changes in mRNA expression
of ATOH1

| | | | | Fold of |
|---|---|---|---|---|
| | | | | changes in |
| | | | | relative |
| | | | | mRNA |
| | | | | expression |
| saRNA | Active target sequence (5′-3′) | Sense sequence (5′-3′) | Antisense sequence (5′-3′) | of ATOH1 |
| RAG9-578 | GCGGCCTTCTCTCTGCTTT | GCGGCCUUCUCUCUGCUUUTT | AAAGCAGAGAGAAGGCCGCTT | 5.88 |
| | (SEQ ID NO: 280) | (SEQ ID NO: 6) | (SEQ ID NO: 143) | |
| RAG9-52 | CAGAAGAGACCCGGAAAGG | CAGAAGAGACCCGGAAAGGTT | CCUUUCCGGGUCUCUUCUGTT | 4.17 |
| | (SEQ ID NO: 281) | (SEQ ID NO: 7) | (SEQ ID NO: 144) | |
| RAG9-139 | CCGGAGAAGCATAGTTGCA | CCGGAGAAGCAUAGUUGCATT | UGCAACUAUGCUUCUCCGGTT | 3.92 |
| | (SEQ ID NO: 282) | (SEQ ID NO: 8) | (SEQ ID NO: 145) | |
| RAG9-120 | CGCGACCTGGTGTGTGATC | CGCGACCUGGUGUGUGAUCTT | GAUCACACACCAGGUCGCGTT | 3.53 |
| | (SEQ ID NO: 283) | (SEQ ID NO: 9) | (SEQ ID NO: 146) | |
| RAG9-339 | GAGCAGGCTTGGGAGTCCT | GAGCAGGCUUGGGAGUCCUTT | AGGACUCCCAAGCCUGCUCTT | 3.41 |
| | (SEQ ID NO: 284) | (SEQ ID NO: 10) | (SEQ ID NO: 147) | |
| RAG9-128 | TAGTTGCACGCGACCTGGT | UAGUUGCACGCGACCUGGUTT | ACCAGGUCGCGUGCAACUATT | 3.37 |
| | (SEQ ID NO: 285) | (SEQ ID NO: 11) | (SEQ ID NO: 148) | |
| RAG9-536 | GGTCCAGAATCGCCCAGAG | GGUCCAGAAUCGCCCAGAGTT | CUCUGGGCGAUUCUGGACCTT | 3.37 |
| | (SEQ ID NO: 286) | (SEQ ID NO: 12) | (SEQ ID NO: 149) | |
| RAG9-53 | GCAGAAGAGACCCGGAAAG | GCAGAAGAGACCCGGAAAGTT | CUUUCCGGGUCUCUUCUGCTT | 3.21 |
| | (SEQ ID NO: 287) | (SEQ ID NO: 13) | (SEQ ID NO: 150) | |
| RAG9-513 | CCGCGGTCGTGCACATCTG | CCGCGGUCGUGCACAUCUGTT | CAGAUGUGCACGACCGCGGTT | 3.19 |
| | (SEQ ID NO: 288) | (SEQ ID NO: 14) | (SEQ ID NO: 151) | |
| RAG9-538 | CGGGTCCAGAATCGCCCAG | CGGGUCCAGAAUCGCCCAGTT | CUGGGCGAUUCUGGACCCGTT | 3.18 |
| | (SEQ ID NO: 289) | (SEQ ID NO: 15) | (SEQ ID NO: 152) | |
| RAG9-150 | GCCTTCAGCAACCGGAGAA | GCCUUCAGCAACCGGAGAATT | UUCUCCGGUUGCUGAAGGCTT | 2.83 |
| | (SEQ ID NO: 290) | (SEQ ID NO: 16) | (SEQ ID NO: 153) | |
| RAG9-418 | ACACCGGAGTCGAATTACA | ACACCGGAGUCGAAUUACATT | UGUAAUUCGACUCCGGUGUTT | 2.81 |
| | (SEQ ID NO: 291) | (SEQ ID NO: 17) | (SEQ ID NO: 154) | |
| RAG9-309 | AACTTTTCTCGGGGTGTAA | AACUUUUCUCGGGGUGUAATT | UUACACCCCGAGAAAAGUUTT | 2.79 |
| | (SEQ ID NO: 292) | (SEQ ID NO: 18) | (SEQ ID NO: 155) | |
| RAG9-331 | TTGGGAGTCCTCTGCACAC | UUGGGAGUCCUCUGCACACTT | GUGUGCAGAGGACUCCCAATT | 2.79 |
| | (SEQ ID NO: 293) | (SEQ ID NO: 19) | (SEQ ID NO: 156) | |
| RAG9-482 | TTGGGCACCAGCCGAGAGC | UUGGGCACCAGCCGAGAGCTT | GCUCUCGGCUGGUGCCCAATT | 2.66 |
| | (SEQ ID NO: 294) | (SEQ ID NO: 20) | (SEQ ID NO: 157) | |
| RAG9-54 | TGCAGAAGAGACCCGGAAA | UGCAGAAGAGACCCGGAAATT | UUUCCGGGUCUCUUCUGCATT | 2.61 |
| | (SEQ ID NO: 295) | (SEQ ID NO: 21) | (SEQ ID NO: 158) | |
| RAG9-533 | CCAGAATCGCCCAGAGCCG | CCAGAAUCGCCCAGAGCCGTT | CGGCUCUGGGCGAUUCUGGTT | 2.57 |
| | (SEQ ID NO: 296) | (SEQ ID NO: 22) | (SEQ ID NO: 159) | |
| RAG9-158 | GGCGCAGCGCCTTCAGCAA | GGCGCAGCGCCUUCAGCAATT | UUGCUGAAGGCGCUGCGCCTT | 2.47 |
| | (SEQ ID NO: 297) | (SEQ ID NO: 23) | (SEQ ID NO: 160) | |
| RAG9-122 | CACGCGACCTGGTGTGTGA | CACGCGACCUGGUGUGUGATT | UCACACACCAGGUCGCGUGTT | 2.26 |
| | (SEQ ID NO: 298) | (SEQ ID NO: 24) | (SEQ ID NO: 161) | |
| RAG9-539 | GCGGGTCCAGAATCGCCCA | GCGGGUCCAGAAUCGCCCATT | UGGGCGAUUCUGGACCCGCTT | 2.21 |
| | (SEQ ID NO: 299) | (SEQ ID NO: 25) | (SEQ ID NO: 162) | |
| RAG9-572 | TTCTCTCTGCTTTGAGTGG | UUCUCUCUGCUUUGAGUGGTT | CCACUCAAAGCAGAGAGAATT | 2.21 |
| | (SEQ ID NO: 300) | (SEQ ID NO: 26) | (SEQ ID NO: 163) | |
| RAG9-473 | AGCCGAGAGCCGGCTCCGC | AGCCGAGAGCCGGCUCCGCTT | GCGGAGCCGGCUCUCGGCUTT | 2.19 |
| | (SEQ ID NO: 301) | (SEQ ID NO: 27) | (SEQ ID NO: 164) | |
| RAG9-502 | CACATCTGACCCGAGTCAG | CACAUCUGACCCGAGUCAGTT | CUGACUCGGGUCAGAUGUGTT | 2.11 |
| | (SEQ ID NO: 302) | (SEQ ID NO: 28) | (SEQ ID NO: 165) | |
| RAG9-472 | GCCGAGAGCCGGCTCCGCA | GCCGAGAGCCGGCUCCGCATT | UGCGGAGCCGGCUCUCGGCTT | 2.11 |
| | (SEQ ID NO: 303) | (SEQ ID NO: 29) | (SEQ ID NO: 166) | |
| RAG9-484 | GCTTGGGCACCAGCCGAGA | GCUUGGGCACCAGCCGAGATT | UCUCGGCUGGUGCCCAAGCTT | 2.10 |
| | (SEQ ID NO: 304) | (SEQ ID NO: 30) | (SEQ ID NO: 167) | |
| RAG9-283 | TTGATTGGCTGCTCGCACG | UUGAUUGGCUGCUCGCACGTT | CGUGCGAGCAGCCAAUCAATT | 2.09 |
| | (SEQ ID NO: 305) | (SEQ ID NO: 31) | (SEQ ID NO: 168) | |
| RAG9-434 | GCCGGGGTGGTGACACACA | GCCGGGGUGGUGACACACATT | UGUGUGUCACCACCCCGGCTT | 2.09 |
| | (SEQ ID NO: 306) | (SEQ ID NO: 32) | (SEQ ID NO: 169) | |
| RAG9-251 | CCTCCATTGGCTGAGAAGA | CCUCCAUUGGCUGAGAAGATT | UCUUCUCAGCCAAUGGAGGTT | 2.09 |
| | (SEQ ID NO: 307) | (SEQ ID NO: 33) | (SEQ ID NO: 170) | |
| RAG9-147 | TTCAGCAACCGGAGAAGCA | UUCAGCAACCGGAGAAGCATT | UGCUUCUCCGGUUGCUGAATT | 2.07 |
| | (SEQ ID NO: 308) | (SEQ ID NO: 34) | (SEQ ID NO: 171) | |
| RAG9-149 | CCTTCAGCAACCGGAGAAG | CCUUCAGCAACCGGAGAAGTT | CUUCUCCGGUUGCUGAAGGTT | 2.07 |
| | (SEQ ID NO: 309) | (SEQ ID NO: 35) | (SEQ ID NO: 172) | |
| RAG9-337 | GCAGGCTTGGGAGTCCTCT | GCAGGCUUGGGAGUCCUCUTT | AGAGGACUCCCAAGCCUGCTT | 2.05 |
| | (SEQ ID NO: 310) | (SEQ ID NO: 36) | (SEQ ID NO: 173) | |
| RAG9-338 | AGCAGGCTTGGGAGTCCTC | AGCAGGCUUGGGAGUCCUCTT | GAGGACUCCCAAGCCUGCUTT | 2.01 |
| | (SEQ ID NO: 311) | (SEQ ID NO: 37) | (SEQ ID NO: 174) | |
| RAG9-485 | AGCTTGGGCACCAGCCGAG | AGCUUGGGCACCAGCCGAGTT | CUCGGCUGGUGCCCAAGCUTT | 1.98 |
| | (SEQ ID NO: 312) | (SEQ ID NO: 38) | (SEQ ID NO: 175) | |
| RAG9-336 | CAGGCTTGGGAGTCCTCTG | CAGGCUUGGGAGUCCUCUGTT | CAGAGGACUCCCAAGCCUGTT | 1.92 |
| | (SEQ ID NO: 313) | (SEQ ID NO: 39) | (SEQ ID NO: 176) | |
| RAG9-503 | GCACATCTGACCCGAGTCA | GCACAUCUGACCCGAGUCATT | UGACUCGGGUCAGAUGUGCTT | 1.90 |
| | (SEQ ID NO: 314) | (SEQ ID NO: 40) | (SEQ ID NO: 177) | |
| RAG9-152 | GCGCCTTCAGCAACCGGAG | GCGCCUUCAGCAACCGGAGTT | CUCCGGUUGCUGAAGGCGCTT | 1.86 |
| | (SEQ ID NO: 315) | (SEQ ID NO: 41) | (SEQ ID NO: 178) | |
| RAG9-116 | ACCTGGTGTGTGATCTCCG | ACCUGGUGUGUGAUCUCCGTT | CGGAGAUCACACACCAGGUTT | 1.85 |
| | (SEQ ID NO: 316) | (SEQ ID NO: 42) | (SEQ ID NO: 179) | |
| RAG9-408 | CGAATTACAGCCCTGCAAT | CGAAUUACAGCCCUGCAAUTT | AUUGCAGGGCUGUAAUUCGTT | 1.84 |
| | (SEQ ID NO: 317) | (SEQ ID NO: 43) | (SEQ ID NO: 180) | |
| RAG9-409 | TCGAATTACAGCCCTGCAA | UCGAAUUACAGCCCUGCAATT | UUGCAGGGCUGUAAUUCGATT | 1.83 |
| | (SEQ ID NO: 318) | (SEQ ID NO: 44) | (SEQ ID NO: 181) | |
| RAG9-317 | CACACAAGAACTTTTCTCG | CACACAAGAACUUUUCUCGTT | CGAGAAAAGUUCUUGUGUGTT | 1.81 |
| | (SEQ ID NO: 319) | (SEQ ID NO: 45) | (SEQ ID NO: 182) | |
| RAG9-330 | TGGGAGTCCTCTGCACACA | UGGGAGUCCUCUGCACACATT | UGUGUGCAGAGGACUCCCATT | 1.80 |
| | (SEQ ID NO: 320) | (SEQ ID NO: 46) | (SEQ ID NO: 183) | |
| RAG9-616 | GGGAGCTGTGATCCTAGCT | GGGAGCUGUGAUCCUAGCUTT | AGCUAGGAUCACAGCUCCCTT | 1.75 |
| | (SEQ ID NO: 321) | (SEQ ID NO: 47) | (SEQ ID NO: 184) | |
| RAG9-151 | CGCCTTCAGCAACCGGAGA | CGCCUUCAGCAACCGGAGATT | UCUCCGGUUGCUGAAGGCGTT | 1.69 |
| | (SEQ ID NO: 322) | (SEQ ID NO: 48) | (SEQ ID NO: 185) | |
| RAG9-615 | GGAGCTGTGATCCTAGCTT | GGAGCUGUGAUCCUAGCUUTT | AAGCUAGGAUCACAGCUCCTT | 1.68 |
| | (SEQ ID NO: 323) | (SEQ ID NO: 49) | (SEQ ID NO: 186) | |
| RAG9-412 | GAGTCGAATTACAGCCCTG | GAGUCGAAUUACAGCCCUGTT | CAGGGCUGUAAUUCGACUCTT | 1.67 |
| | (SEQ ID NO: 324) | (SEQ ID NO: 50) | (SEQ ID NO: 187) | |
| RAG9-162 | AAGGGGCGCAGCGCCTTCA | AAGGGGCGCAGCGCCUUCATT | UGAAGGCGCUGCGCCCCUUTT | 1.66 |
| | (SEQ ID NO: 325) | (SEQ ID NO: 51) | (SEQ ID NO: 188) | |
| RAG9-417 | CACCGGAGTCGAATTACAG | CACCGGAGUCGAAUUACAGTT | CUGUAAUUCGACUCCGGUGTT | 1.64 |
| | (SEQ ID NO: 326) | (SEQ ID NO: 52) | (SEQ ID NO: 189) | |
| RAG9-324 | TCCTCTGCACACAAGAACT | UCCUCUGCACACAAGAACUTT | AGUUCUUGUGUGCAGAGGATT | 1.61 |
| | (SEQ ID NO: 327) | (SEQ ID NO: 53) | (SEQ ID NO: 190) | |
| RAG9-455 | CACCGCTCCCGCACCCCAG | CACCGCUCCCGCACCCCAGTT | CUGGGGUGCGGGAGCGGUGTT | 1.61 |
| | (SEQ ID NO: 328) | (SEQ ID NO: 54) | (SEQ ID NO: 191) | |
| RAG9-325 | GTCCTCTGCACACAAGAAC | GUCCUCUGCACACAAGAACTT | GUUCUUGUGUGCAGAGGACTT | 1.61 |
| | (SEQ ID NO: 329) | (SEQ ID NO: 55) | (SEQ ID NO: 192) | |
| RAG9-501 | ACATCTGACCCGAGTCAGC | ACAUCUGACCCGAGUCAGCTT | GCUGACUCGGGUCAGAUGUTT | 1.61 |
| | (SEQ ID NO: 330) | (SEQ ID NO: 56) | (SEQ ID NO: 193) | |
| RAG9-577 | CGGCCTTCTCTCTGCTTTG | CGGCCUUCUCUCUGCUUUGTT | CAAAGCAGAGAGAAGGCCGTT | 1.60 |
| | (SEQ ID NO: 331) | (SEQ ID NO: 57) | (SEQ ID NO: 194) | |
| RAG9-191 | GGAGACTGAGTGGCGCGTG | GGAGACUGAGUGGCGCGUGTT | CACGCGCCACUCAGUCUCCTT | 1.57 |
| | (SEQ ID NO: 332) | (SEQ ID NO: 58) | (SEQ ID NO: 195) | |
| RAG9-148 | CTTCAGCAACCGGAGAAGC | CUUCAGCAACCGGAGAAGCTT | GCUUCUCCGGUUGCUGAAGTT | 1.56 |
| | (SEQ ID NO: 333) | (SEQ ID NO: 59) | (SEQ ID NO: 196) | |
| RAG9-528 | ATCGCCCAGAGCCGCCCGC | AUCGCCCAGAGCCGCCCGCTT | GCGGGCGGCUCUGGGCGAUTT | 1.56 |
| | (SEQ ID NO: 334) | (SEQ ID NO: 60) | (SEQ ID NO: 197) | |
| RAG9-552 | TTCTGGGCGCCTGGCGGGT | UUCUGGGCGCCUGGCGGGUTT | ACCCGCCAGGCGCCCAGAATT | 1.55 |
| | (SEQ ID NO: 335) | (SEQ ID NO: 61) | (SEQ ID NO: 198) | |
| RAG9-279 | TTGGCTGCTCGCACGCGCC | UUGGCUGCUCGCACGCGCCTT | GGCGCGUGCGAGCAGCCAATT | 1.53 |
| | (SEQ ID NO: 336) | (SEQ ID NO: 62) | (SEQ ID NO: 199) | |
| RAG9-483 | CTTGGGCACCAGCCGAGAG | CUUGGGCACCAGCCGAGAGTT | CUCUCGGCUGGUGCCCAAGTT | 1.52 |
| | (SEQ ID NO: 337) | (SEQ ID NO: 63) | (SEQ ID NO: 200) | |
| RAG9-628 | GGCATCAGAAGTGGGAGCT | GGCAUCAGAAGUGGGAGCUTT | AGCUCCCACUUCUGAUGCCTT | 1.52 |
| | (SEQ ID NO: 338) | (SEQ ID NO: 64) | (SEQ ID NO: 201) | |
| RAG9-540 | GGCGGGTCCAGAATCGCCC | GGCGGGUCCAGAAUCGCCCTT | GGGCGAUUCUGGACCCGCCTT | 1.50 |
| | (SEQ ID NO: 339) | (SEQ ID NO: 65) | (SEQ ID NO: 202) | |
| RAG9-626 | CATCAGAAGTGGGAGCTGT | CAUCAGAAGUGGGAGCUGUTT | ACAGCUCCCACUUCUGAUGTT | 1.50 |
| | (SEQ ID NO: 340) | (SEQ ID NO: 66) | (SEQ ID NO: 203) | |
| RAG9-515 | GCCCGCGGTCGTGCACATC | GCCCGCGGUCGUGCACAUCTT | GAUGUGCACGACCGCGGGCTT | 1.49 |
| | (SEQ ID NO: 341) | (SEQ ID NO: 67) | (SEQ ID NO: 204) | |
| RAG9-404 | TTACAGCCCTGCAATTAAC | UUACAGCCCUGCAAUUAACTT | GUUAAUUGCAGGGCUGUAATT | 1.49 |
| | (SEQ ID NO: 342) | (SEQ ID NO: 68) | (SEQ ID NO: 205) | |
| RAG9-55 | TTGCAGAAGAGACCCGGAA | UUGCAGAAGAGACCCGGAATT | UUCCGGGUCUCUUCUGCAATT | 1.49 |
| | (SEQ ID NO: 343) | (SEQ ID NO: 69) | (SEQ ID NO: 206) | |
| RAG9-161 | AGGGGCGCAGCGCCTTCAG | AGGGGCGCAGCGCCUUCAGTT | CUGAAGGCGCUGCGCCCCUTT | 1.48 |
| | (SEQ ID NO: 344) | (SEQ ID NO: 70) | (SEQ ID NO: 207) | |
| RAG9-322 | CTCTGCACACAAGAACTTT | CUCUGCACACAAGAACUUUTT | AAAGUUCUUGUGUGCAGAGTT | 1.47 |
| | (SEQ ID NO: 345) | (SEQ ID NO: 71) | (SEQ ID NO: 208) | |
| RAG9-106 | TGATCTCCGAGTGGGTGGG | UGAUCUCCGAGUGGGUGGGTT | CCCACCCACUCGGAGAUCATT | 1.46 |
| | (SEQ ID NO: 346) | (SEQ ID NO: 72) | (SEQ ID NO: 209) | |
| RAG9-56 | GTTGCAGAAGAGACCCGGA | GUUGCAGAAGAGACCCGGATT | UCCGGGUCUCUUCUGCAACTT | 1.46 |
| | (SEQ ID NO: 347) | (SEQ ID NO: 73) | (SEQ ID NO: 210) | |
| RAG9-320 | CTGCACACAAGAACTTTTC | CUGCACACAAGAACUUUUCTT | GAAAAGUUCUUGUGUGCAGTT | 1.44 |
| | (SEQ ID NO: 348) | (SEQ ID NO: 74) | (SEQ ID NO: 211) | |
| RAG9-491 | CGAGTCAGCTTGGGCACCA | CGAGUCAGCUUGGGCACCATT | UGGUGCCCAAGCUGACUCGTT | 1.43 |
| | (SEQ ID NO: 349) | (SEQ ID NO: 75) | (SEQ ID NO: 212) | |
| RAG9-488 | GTCAGCTTGGGCACCAGCC | GUCAGCUUGGGCACCAGCCTT | GGCUGGUGCCCAAGCUGACTT | 1.43 |
| | (SEQ ID NO: 350) | (SEQ ID NO: 76) | (SEQ ID NO: 213) | |
| RAG9-413 | GGAGTCGAATTACAGCCCT | GGAGUCGAAUUACAGCCCUTT | AGGGCUGUAAUUCGACUCCTT | 1.42 |
| | (SEQ ID NO: 351) | (SEQ ID NO: 77) | (SEQ ID NO: 214) | |
| RAG9-512 | CGCGGTCGTGCACATCTGA | CGCGGUCGUGCACAUCUGATT | UCAGAUGUGCACGACCGCGTT | 1.42 |
| | (SEQ ID NO: 352) | (SEQ ID NO: 78) | (SEQ ID NO: 215) | |
| RAG9-113 | TGGTGTGTGATCTCCGAGT | UGGUGUGUGAUCUCCGAGUTT | ACUCGGAGAUCACACACCATT | 1.42 |
| | (SEQ ID NO: 353) | (SEQ ID NO: 79) | (SEQ ID NO: 216) | |
| RAG9-274 | TGCTCGCACGCGCCTGCCC | UGCUCGCACGCGCCUGCCCTT | GGGCAGGCGCGUGCGAGCATT | 1.41 |
| | (SEQ ID NO: 354) | (SEQ ID NO: 80) | (SEQ ID NO: 217) | |
| RAG9-634 | TGCTTGGGCATCAGAAGTG | UGCUUGGGCAUCAGAAGUGTT | CACUUCUGAUGCCCAAGCATT | 1.39 |
| | (SEQ ID NO: 355) | (SEQ ID NO: 81) | (SEQ ID NO: 218) | |
| RAG9-558 | AGTGGCTTCTGGGCGCCTG | AGUGGCUUCUGGGCGCCUGTT | CAGGCGCCCAGAAGCCACUTT | 1.39 |
| | (SEQ ID NO: 356) | (SEQ ID NO: 82) | (SEQ ID NO: 219) | |
| RAG9-326 | AGTCCTCTGCACACAAGAA | AGUCCUCUGCACACAAGAATT | UUCUUGUGUGCAGAGGACUTT | 1.38 |
| | (SEQ ID NO: 357) | (SEQ ID NO: 83) | (SEQ ID NO: 220) | |
| RAG9-110 | TGTGTGATCTCCGAGTGGG | UGUGUGAUCUCCGAGUGGGTT | CCCACUCGGAGAUCACACATT | 1.37 |
| | (SEQ ID NO: 358) | (SEQ ID NO: 84) | (SEQ ID NO: 221) | |
| RAG9-340 | TGAGCAGGCTTGGGAGTCC | UGAGCAGGCUUGGGAGUCCTT | GGACUCCCAAGCCUGCUCATT | 1.37 |
| | (SEQ ID NO: 359) | (SEQ ID NO: 85) | (SEQ ID NO: 222) | |
| RAG9-506 | CGTGCACATCTGACCCGAG | CGUGCACAUCUGACCCGAGTT | CUCGGGUCAGAUGUGCACGTT | 1.36 |
| | (SEQ ID NO: 360) | (SEQ ID NO: 86) | (SEQ ID NO: 223) | |
| RAG9-507 | TCGTGCACATCTGACCCGA | UCGUGCACAUCUGACCCGATT | UCGGGUCAGAUGUGCACGATT | 1.36 |
| | (SEQ ID NO: 361) | (SEQ ID NO: 87) | (SEQ ID NO: 224) | |
| RAG9- | AGCTTCTGATTGGCTCTTT | AGCUUCUGAUUGGCUCUUUTT | AAAGAGCCAAUCAGAAGCUTT | 1.35 |
| 1407 | (SEQ ID NO: 362) | (SEQ ID NO: 88) | (SEQ ID NO: 225) | |
| RAG9-508 | GTCGTGCACATCTGACCCG | GUCGUGCACAUCUGACCCGTT | CGGGUCAGAUGUGCACGACTT | 1.35 |
| | (SEQ ID NO: 363) | (SEQ ID NO: 89) | (SEQ ID NO: 226) | |
| RAG9-588 | CACAGGGTAGGCGGCCTTC | CACAGGGUAGGCGGCCUUCTT | GAAGGCCGCCUACCCUGUGTT | 1.35 |
| | (SEQ ID NO: 364) | (SEQ ID NO: 90) | (SEQ ID NO: 227) | |
| RAG9-114 | CTGGTGTGTGATCTCCGAG | CUGGUGUGUGAUCUCCGAGTT | CUCGGAGAUCACACACCAGTT | 1.34 |
| | (SEQ ID NO: 365) | (SEQ ID NO: 91) | (SEQ ID NO: 228) | |
| RAG9-456 | GCACCGCTCCCGCACCCCA | GCACCGCUCCCGCACCCCATT | UGGGGUGCGGGAGCGGUGCTT | 1.34 |
| | (SEQ ID NO: 366) | (SEQ ID NO: 92) | (SEQ ID NO: 229) | |
| RAG9-312 | AAGAACTTTTCTCGGGGTG | AAGAACUUUUCUCGGGGUGTT | CACCCCGAGAAAAGUUCUUTT | 1.34 |
| | (SEQ ID NO: 367) | (SEQ ID NO: 93) | (SEQ ID NO: 230) | |
| RAG9-315 | CACAAGAACTTTTCTCGGG | CACAAGAACUUUUCUCGGGTT | CCCGAGAAAAGUUCUUGUGTT | 1.33 |
| | (SEQ ID NO: 368) | (SEQ ID NO: 94) | (SEQ ID NO: 231) | |
| RAG9-575 | GCCTTCTCTCTGCTTTGAG | GCCUUCUCUCUGCUUUGAGTT | CUCAAAGCAGAGAGAAGGCTT | 1.32 |
| | (SEQ ID NO: 369) | (SEQ ID NO: 95) | (SEQ ID NO: 232) | |
| RAG9-467 | GAGCCGGCTCCGCACCGCT | GAGCCGGCUCCGCACCGCUTT | AGCGGUGCGGAGCCGGCUCTT | 1.32 |
| | (SEQ ID NO: 370) | (SEQ ID NO: 96) | (SEQ ID NO: 233) | |
| RAG9-231 | ACGCGACCGGCGCGAGGAG | ACGCGACCGGCGCGAGGAGTT | CUCCUCGCGCCGGUCGCGUTT | 1.31 |
| | (SEQ ID NO: 371) | (SEQ ID NO: 97) | (SEQ ID NO: 234) | |
| RAG9-637 | AAATGCTTGGGCATCAGAA | AAAUGCUUGGGCAUCAGAATT | UUCUGAUGCCCAAGCAUUUTT | 1.31 |
| | (SEQ ID NO: 372) | (SEQ ID NO: 98) | (SEQ ID NO: 235) | |
| RAG9-633 | GCTTGGGCATCAGAAGTGG | GCUUGGGCAUCAGAAGUGGTT | CCACUUCUGAUGCCCAAGCTT | 1.30 |
| | (SEQ ID NO: 373) | (SEQ ID NO: 99) | (SEQ ID NO: 236) | |
| RAG9-519 | AGCCGCCCGCGGTCGTGCA | AGCCGCCCGCGGUCGUGCATT | UGCACGACCGCGGGCGGCUTT | 1.30 |
| | (SEQ ID NO: 374) | (SEQ ID NO: 100) | (SEQ ID NO: 237) | |
| RAG9-509 | GGTCGTGCACATCTGACCC | GGUCGUGCACAUCUGACCCTT | GGGUCAGAUGUGCACGACCTT | 1.29 |
| | (SEQ ID NO: 375) | (SEQ ID NO: 101) | (SEQ ID NO: 238) | |
| RAG9-146 | TCAGCAACCGGAGAAGCAT | UCAGCAACCGGAGAAGCAUTT | AUGCUUCUCCGGUUGCUGATT | 1.29 |
| | (SEQ ID NO: 376) | (SEQ ID NO: 102) | (SEQ ID NO: 239) | |
| RAG9-531 | AGAATCGCCCAGAGCCGCC | AGAAUCGCCCAGAGCCGCCTT | GGCGGCUCUGGGCGAUUCUTT | 1.29 |
| | (SEQ ID NO: 377) | (SEQ ID NO: 103) | (SEQ ID NO: 240) | |
| RAG9-614 | GAGCTGTGATCCTAGCTTG | GAGCUGUGAUCCUAGCUUGTT | CAAGCUAGGAUCACAGCUCTT | 1.29 |
| | (SEQ ID NO: 378) | (SEQ ID NO: 104) | (SEQ ID NO: 241) | |
| RAG9-138 | CGGAGAAGCATAGTTGCAC | CGGAGAAGCAUAGUUGCACTT | GUGCAACUAUGCUUCUCCGTT | 1.29 |
| | (SEQ ID NO: 379) | (SEQ ID NO: 105) | (SEQ ID NO: 242) | |
| RAG9-129 | ATAGTTGCACGCGACCTGG | AUAGUUGCACGCGACCUGGTT | CCAGGUCGCGUGCAACUAUTT | 1.28 |
| | (SEQ ID NO: 380) | (SEQ ID NO: 106) | (SEQ ID NO: 243) | |
| RAG9-308 | ACTTTTCTCGGGGTGTAAA | ACUUUUCUCGGGUGUAAATT | UUUACACCCCGAGAAAAGUTT | 1.28 |
| | (SEQ ID NO: 381) | (SEQ ID NO: 107) | (SEQ ID NO: 244) | |
| RAG9-553 | CTTCTGGGCGCCTGGCGGG | CUUCUGGGCGCCUGGCGGGTT | CCCGCCAGGCGCCCAGAAGTT | 1.27 |
| | (SEQ ID NO: 382) | (SEQ ID NO: 108) | (SEQ ID NO: 245) | |
| RAG9-439 | CAGCCGCCGGGGTGGTGAC | CAGCCGCCGGGGUGGUGACTT | GUCACCACCCCGGCGGCUGTT | 1.27 |
| | (SEQ ID NO: 383) | (SEQ ID NO: 109) | (SEQ ID NO: 246) | |
| RAG9-613 | AGCTGTGATCCTAGCTTGG | AGCUGUGAUCCUAGCUUGGTT | CCAAGCUAGGAUCACAGCUTT | 1.26 |
| | (SEQ ID NO: 384) | (SEQ ID NO: 110) | (SEQ ID NO: 247) | |
| RAG9-119 | GCGACCTGGTGTGTGATCT | GCGACCUGGUGUGUGAUCUTT | AGAUCACACACCAGGUCGCTT | 1.25 |
| | (SEQ ID NO: 385) | (SEQ ID NO: 111) | (SEQ ID NO: 248) | |
| RAG9-590 | AGCACAGGGTAGGCGGCCT | AGCACAGGGUAGGCGGCCUTT | AGGCCGCCUACCCUGUGCUTT | 1.25 |
| | (SEQ ID NO: 386) | (SEQ ID NO: 112) | (SEQ ID NO: 249) | |
| RAG9-546 | GCGCCTGGCGGGTCCAGAA | GCGCCUGGCGGGUCCAGAATT | UUCUGGACCCGCCAGGCGCTT | 1.25 |
| | (SEQ ID NO: 387) | (SEQ ID NO: 113) | (SEQ ID NO: 250) | |
| RAG9-627 | GCATCAGAAGTGGGAGCTG | GCAUCAGAAGUGGGAGCUGTT | CAGCUCCCACUUCUGAUGCTT | 1.24 |
| | (SEQ ID NO: 388) | (SEQ ID NO: 114) | (SEQ ID NO: 251) | |
| RAG9-530 | GAATCGCCCAGAGCCGCCC | GAAUCGCCCAGAGCCGCCCTT | GGGCGGCUCUGGGCGAUUCTT | 1.24 |
| | (SEQ ID NO: 389) | (SEQ ID NO: 115) | (SEQ ID NO: 252) | |
| RAG9-159 | GGGCGCAGCGCCTTCAGCA | GGGCGCAGCGCCUUCAGCATT | UGCUGAAGGCGCUGCGCCCTT | 1.24 |
| | (SEQ ID NO: 390) | (SEQ ID NO: 116) | (SEQ ID NO: 253) | |
| RAG9-403 | TACAGCCCTGCAATTAACA | UACAGCCCUGCAAUUAACATT | UGUUAAUUGCAGGGCUGUATT | 1.23 |
| | (SEQ ID NO: 391) | (SEQ ID NO: 117) | (SEQ ID NO: 254) | |
| RAG9-388 | AACATATGAATCTGACGAA | AACAUAUGAAUCUGACGAATT | UUCGUCAGAUUCAUAUGUUTT | 1.22 |
| | (SEQ ID NO: 392) | (SEQ ID NO: 118) | (SEQ ID NO: 255) | |
| RAG9-454 | ACCGCTCCCGCACCCCAGC | ACCGCUCCCGCACCCCAGCTT | GCUGGGGUGCGGGAGCGGUTT | 1.22 |
| | (SEQ ID NO: 393) | (SEQ ID NO: 119) | (SEQ ID NO: 256) | |
| RAG9-189 | AGACTGAGTGGCGCGTGCC | AGACUGAGUGGCGCGUGCCTT | GGCACGCGCCACUCAGUCUTT | 1.21 |
| | (SEQ ID NO: 394) | (SEQ ID NO: 120) | (SEQ ID NO: 257) | |
| RAG9-131 | GCATAGTTGCACGCGACCT | GCAUAGUUGCACGCGACCUTT | AGGUCGCGUGCAACUAUGCTT | 1.20 |
| | (SEQ ID NO: 395) | (SEQ ID NO: 121) | (SEQ ID NO: 258) | |
| RAG9-504 | TGCACATCTGACCCGAGTC | UGCACAUCUGACCCGAGUCTT | GACUCGGGUCAGAUGUGCATT | 1.19 |
| | (SEQ ID NO: 396) | (SEQ ID NO: 122) | (SEQ ID NO: 259) | |
| RAG9-529 | AATCGCCCAGAGCCGCCCG | AAUCGCCCAGAGCCGCCCGTT | CGGGCGGCUCUGGGCGAUUTT | 1.19 |
| | (SEQ ID NO: 397) | (SEQ ID NO: 123) | (SEQ ID NO: 260) | |
| RAG9-493 | CCCGAGTCAGCTTGGGCAC | CCCGAGUCAGCUUGGGCACTT | GUGCCCAAGCUGACUCGGGTT | 1.19 |
| | (SEQ ID NO: 398) | (SEQ ID NO: 124) | (SEQ ID NO: 261) | |
| RAG9-481 | TGGGCACCAGCCGAGAGCC | UGGGCACCAGCCGAGAGCCTT | GGCUCUCGGCUGGUGCCCATT | 1.18 |
| | (SEQ ID NO: 399) | (SEQ ID NO: 125) | (SEQ ID NO: 262) | |
| RAG9-307 | CTTTTCTCGGGGTGTAAAA | CUUUUCUCGGGGUGUAAAATT | UUUUACACCCCGAGAAAAGTT | 1.16 |
| | (SEQ ID NO: 400) | (SEQ ID NO: 126) | (SEQ ID NO: 263) | |
| RAG9-564 | GCTTTGAGTGGCTTCTGGG | GCUUUGAGUGGCUUCUGGGTT | CCCAGAAGCCACUCAAAGCTT | 1.16 |
| | (SEQ ID NO: 401) | (SEQ ID NO: 127) | (SEQ ID NO: 264) | |
| RAG9-410 | GTCGAATTACAGCCCTGCA | GUCGAAUUACAGCCCUGCATT | UGCAGGGCUGUAAUUCGACTT | 1.15 |
| | (SEQ ID NO: 402) | (SEQ ID NO: 128) | (SEQ ID NO: 265) | |
| RAG9-511 | GCGGTCGTGCACATCTGAC | GCGGUCGUGCACAUCUGACTT | GUCAGAUGUGCACGACCGCTT | 1.15 |
| | (SEQ ID NO: 403) | (SEQ ID NO: 129) | (SEQ ID NO: 266) | |
| RAG9-406 | AATTACAGCCCTGCAATTA | AAUUACAGCCCUGCAAUUATT | UAAUUGCAGGGCUGUAAUUTT | 1.15 |
| | (SEQ ID NO: 404) | (SEQ ID NO: 130) | (SEQ ID NO: 267) | |
| RAG9-58 | ACGTTGCAGAAGAGACCCG | ACGUUGCAGAAGAGACCCGTT | CGGGUCUCUUCUGCAACGUTT | 1.15 |
| | (SEQ ID NO: 405) | (SEQ ID NO: 131) | (SEQ ID NO: 268) | |
| RAG9-547 | GGCGCCTGGCGGGTCCAGA | GGCGCCUGGCGGGUCCAGATT | UCUGGACCCGCCAGGCGCCTT | 1.15 |
| | (SEQ ID NO: 406) | (SEQ ID NO: 132) | (SEQ ID NO: 269) | |
| RAG9-123 | GCACGCGACCTGGTGTGTG | GCACGCGACCUGGUGUGUGTT | CACACACCAGGUCGCGUGCTT | 1.14 |
| | (SEQ ID NO: 407) | (SEQ ID NO: 133) | (SEQ ID NO: 270) | |
| RAG9-124 | TGCACGCGACCTGGTGTGT | UGCACGCGACCUGGUGUGUTT | ACACACCAGGUCGCGUGCATT | 1.14 |
| | (SEQ ID NO: 408) | (SEQ ID NO: 134) | (SEQ ID NO: 271) | |
| RAG9-436 | CCGCCGGGGTGGTGACACA | CCGCCGGGGUGGUGACACATT | UGUGUCACCACCCCGGCGGTT | 1.14 |
| | (SEQ ID NO: 409) | (SEQ ID NO: 135) | (SEQ ID NO: 272) | |
| RAG9-286 | TCTTTGATTGGCTGCTCGC | UCUUUGAUUGGCUGCUCGCTT | GCGAGCAGCCAAUCAAAGATT | 1.13 |
| | (SEQ ID NO: 410) | (SEQ ID NO: 136) | (SEQ ID NO: 273) | |
| RAG9-420 | ACACACCGGAGTCGAATTA | ACACACCGGAGUCGAAUUATT | UAAUUCGACUCCGGUGUGUTT | 1.13 |
| | (SEQ ID NO: 411) | (SEQ ID NO: 137) | (SEQ ID NO: 274) | |
| RAG9-636 | AATGCTTGGGCATCAGAAG | AAUGCUUGGGCAUCAGAAGTT | CUUCUGAUGCCCAAGCAUUTT | 1.13 |
| | (SEQ ID NO: 412) | (SEQ ID NO: 138) | (SEQ ID NO: 275) | |
| RAG9-289 | AACTCTTTGATTGGCTGCT | AACUCUUUGAUUGGCUGCUTT | AGCAGCCAAUCAAAGAGUUTT | 1.13 |
| | (SEQ ID NO: 413) | (SEQ ID NO: 139) | (SEQ ID NO: 276) | |
| RAG9-537 | GGGTCCAGAATCGCCCAGA | GGGUCCAGAAUCGCCCAGATT | UCUGGGCGAUUCUGGACCCTT | 1.12 |
| | (SEQ ID NO: 414) | (SEQ ID NO: 140) | (SEQ ID NO: 277) | |
| RAG9-419 | CACACCGGAGTCGAATTAC | CACACCGGAGUCGAAUUACTT | GUAAUUCGACUCCGGUGUGTT | 1.12 |
| | (SEQ ID NO: 415) | (SEQ ID NO: 141) | (SEQ ID NO: 278) | |
| RAG9-327 | GAGTCCTCTGCACACAAGA | GAGUCCUCUGCACACAAGATT | UCUUGUGUGCAGAGGACUCTT | 1.10 |
| | (SEQ ID NO: 416) | (SEQ ID NO: 142) | (SEQ ID NO: 279) | |

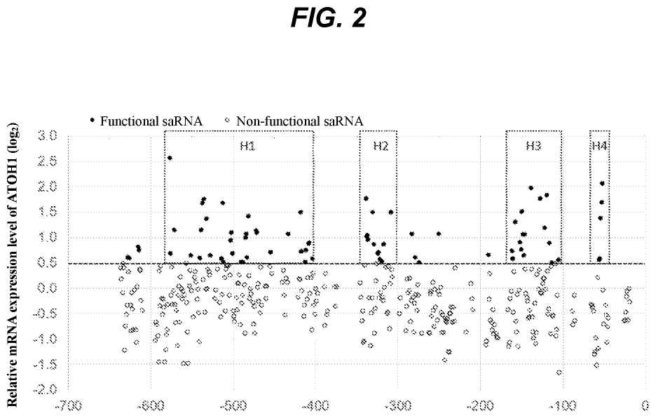

It can be clearly seen from the arrangement of activities of the 403 saRNAs according to their positions within the ATOH1 promoter region that the functional saRNAs are gathered together, that is, in certain promoter regions, the activating saRNAs gathered in specific “hotspot” regions (FIG. 2). As shown in FIG. 2, 4 hotspot regions respectively appeared in region (H1) from −578 to −386, region (H2) from −339 to −291, region (H3) from −162 to −86 and region (H4) from −55 to −34 of the promoter, respectively, showing high gathering of the activating saRNAs, and the proportion of functional saRNAs was 41.0%, 51.6%, 45.6%, and 100%, respectively. These results indicate that the functional saRNAs were not randomly distributed within the promoter, but existed in the specific hotspot regions.

The sequence of the hotspot H1 (5′ to 3′: −578 to −386) corresponds to SEQ ID NO: 2 in the sequence listing:

gcggccttctctctgctttgagtggcttctgggcgcctggcgggtccaga
atcgcccagagccgcccgcggtcgtgcacatctgacccgagtcagcttgg
gcaccagccgagagccggctccgcaccgctcccgcaccccagccgccggg
gtggtgacacacaccggagtcgaattacagccctgcaattaa

The sequence of the hotspot H2 (5′ to 3′: −339 to −291) corresponds to SEQ ID NO: 3 in the sequence listing:

tgcacacaagaacttttctcggggtgtaa

The sequence of the hotspot H3 (5′ to 3′: −162 to −86) corresponds to SEQ ID NO: 4 in the sequence listing:

aaggggcgcagcgccttcagcaaccggagaagcatagttgcacgcgacct
ggtgtgtgatctccgagtgggtgggg

The sequence of the hotspot H4 (5′ to 3′: −55 to −34) corresponds to SEQ ID NO: 5 in the sequence listing:

ttgcagaagagacccggaaagg

### Example 3: Validation of High-Throughput Screening Results

Normal Human Epidermal Keratinocytes (NHEK) cells and human renal embryonic cells (HEK293T) were seeded into a 6-well plate with 2×10⁵ to 3×10⁵ cells/well and were reverse transfected with oligonucleotide duplexes. Total cellular RNAs were extracted using an RNeasy Plus Mini kit (Qiagen). The resulting RNA (1 μg) was reverse transcribed into cDNA using a PrimeScript RT kit containing gDNA Eraser (Takara, Shlga, Japan). The resulting cDNA was amplified by qPCR using an ABI 7500 fast real-time PCR system (Applied Biosystems) and SYBR Premix Ex Taq II reagent (Takara, Shlga, Japan). The reaction conditions were: 95° C. for 3 s, 60° C. for 30 s, and 40 cycles. HPRT1 was used as internal control and RAG9-871i as inhibitory dsRNA control. The phase expression value of the ATOH1 gene was calculated by a method of 2^{-ΔΔCT}. All the primer sequences used are listed in Table 3.

In order to calculate the expression value (Eᵣₑₗ) of ATOH1 (target gene) of an saRNA-transfected sample relative to control treatment (mock), the Ct values of the target gene and the internal control genes were substituted into Formula 1 for calculation.

Eᵣₑₗ₌2^{(CtTm-CtTs)}/2^{(CtRm-CtRs)} (Formula 1)

wherein CtTm was the Ct value of the target gene from Mock sample; CtTs was the Ct value of the target gene from the saRNA-treated sample; CtRm was the Ct value of the internal control gene from mock-treated sample; CtRs was the Ct value of the internal control gene from the saRNA-treated sample.

TABLE 3
Primer sequences for two-step qRT-PCR analysis

| Primer | Sequence No. | Sequence (5′-3′) |
|---|---|---|
| ATOH1 F1 | SEQ ID NO: 417 | CCCTTCCAGCAAACAGGTGA |
| ATOH1 R1 | SEQ ID NO: 418 | GAACGACGGGATAACATTGCG |
| HPRT1 F | SEQ ID NO: 419 | ATGGACAGGACTGAACGTCTT |
| HPRT1 R | SEQ ID NO: 420 | TCCAGCAGGTCAGCAAAGAA |

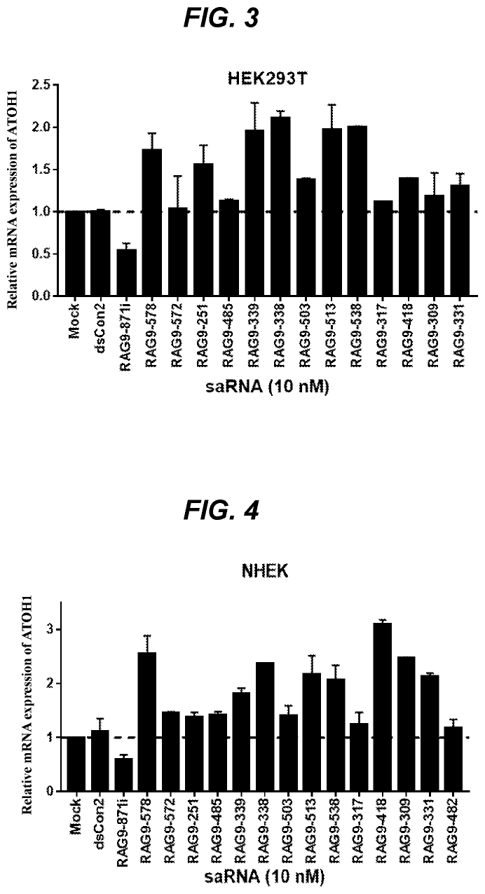

As shown in FIG. 3 and FIG. 4, randomly selected functional saRNAs were verified in HEK293T cells and NHEK cells, and both showed up-regulation of mRNA expression of ATOH1.

TABLE 4
Double-stranded RNA sequences as study controls

| Double- | | |
|---|---|---|
| stranded RNA | Sequence No. | Sequence (5′-3′) |
| dsCon2-sense | SEQ ID NO: 421 | ACUACUGAGUGACAGUAGATT |
| strand | | |
| dsCon2- | SEQ ID NO: 422 | UCUACUGUCACUCAGUAGUTT |
| antisense | | |
| strand | | |
| RAG9-871i- | SEQ ID NO: 423 | GCUCUGCACUUCUCGACUUTT |
| sense strand | | |
| RAG9-871i- | SEQ ID NO: 424 | AAGUCGAGAAGUGCAGAGCTT |
| antisense | | |
| strand | | |

### Example 4. saRNAs Promoting ATOH1 Protein Expression

HEK293T cells were plated into a 96-well plate at a concentration of 3×10³ to 5×10³ cells/well, cultured overnight, and transfected with 7 randomly selected functional saRNAs. Seventy two hours after transfection, cells were collected and lysed using a cell lysis buffer (1×RIPA buffer, CST) which contained a protease inhibitor. Proteins were quantified by BCA method (Thermo) and separated by polyacrylamide gel electrophoresis and conversion to a 0.45 μm PVDF membrane. Primary Rabbit monoclonal anti-ATOH1 (Abcam) and α/β-tubulin antibodies (Cell Signaling Technology) were used to detect blots and a secondary anti-rabbit IgG, HRP-linked antibody (Cell Signaling Technology) was used. Signals were detected using an Image Lab (BIO-RAD, Chemistry Doctm MP imaging system).

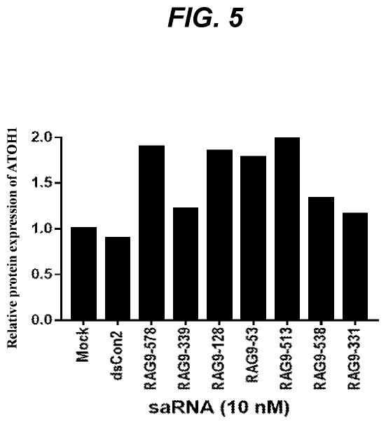

As shown in FIG. 5, the 7 randomly selected activating saRNAs were capable of promoting or increasing the expression of ATOH1 protein.

Based on these results, a plurality of saRNAs capable of activating the expression of ATOH1 gene were found through high-throughput screening of saRNAs targeting ATOH1 gene promoter. The saRNAs can up-regulate the expression of the ATOH1 gene on mRNA and at the protein expression level, and can be used to treat a disease or symptom caused by a decrease or insufficient expression of the ATOH1 protein, or used in the preparation of a method or drug for treating the disease or symptom.

### REFERENCES

1. Aragaki, M., K. Tsuchiya, R. Okamoto, S. Yoshioka, T. Nakamura, N. Sakamoto, T. Kanai, and M. Watanabe. 2008. ‘Proteasomal degradation of Atoh1 by aberrant Wnt signaling maintains the undifferentiated state of colon cancer’, Biochem Biophys Res Commun, 368: 923-9.
2. Bermingham, N. A., B. A. Hassan, S. D. Price, M. A. Vollrath, N. Ben-Arie, R. A. Eatock, H. J. Bellen, A. Lysakowski, and H. Y. Zoghbi. 1999. ‘Math1: an essential gene for the generation of inner ear hair cells’, Science, 284: 1837-41.
3. Gubbels, S. P., D. W. Woessner, J. C. Mitchell, A. J. Ricci, and J. V. Brigande. 2008. ‘Functional auditory hair cells produced in the mammalian cochlea by in utero gene transfer’, Nature, 455: 537-41.
4. Gurtler, N., and A. K. Lalwani. 2002. ‘Etiology of syndromic and nonsyndromic sensorineural hearing loss’, Otolaryngol Clin North Am, 35: 891-908.
5. Izumikawa, M., R. Minoda, K. Kawamoto, K. A. Abrashkin, D. L. Swiderski, D. F. Dolan, D. E. Brough, and Y. Raphael. 2005. ‘Auditory hair cell replacement and hearing improvement by Atoh1 gene therapy in deaf mammals’, Nat Med, 11: 271-6.
6. Kawamoto, K., S. Ishimoto, R. Minoda, D. E. Brough, and Y. Raphael. 2003. ‘Math1 gene transfer generates new cochlear hair cells in mature guinea pigs in vivo’, J Neurosci, 23: 4395-400.
7. Kazanjian A., Shroyer S. F. 2011. NOTCH Signaling and ATOH1 in Colorectal Cancers. Curr Colorectal Cancer Rep June; 7(2):121-127.
8. Leow, C. C., P. Polakis, and W. Q. Gao. 2005. ‘A role for Hath1, a bHLH transcription factor, in colon adenocarcinoma’, Ann N Y Acad Sci, 1059: 174-83.
9. Liberman, M. C. 1990. ‘Quantitative assessment of inner ear pathology following ototoxic drugs or acoustic trauma’, Toxicol Pathol, 18: 138-48.
10. Richardson, R. T., and P. J. Atkinson. 2015. ‘Atoh1 gene therapy in the cochlea for hair cell regeneration’, Expert Opin Biol Ther, 15: 417-30.
11. Zheng, J. L., and W. Q. Gao. 2000. ‘Overexpression of Math1 induces robust production of extra hair cells in postnatal rat inner ears’, Nat Neurosci, 3: 580-6.

1. A small activating nucleic acid molecule, comprising a first nucleic acid strand and a second nucleic acid strand, wherein the first nucleic acid strand has at least 75% homology or complementarity to a continuous sequence of 16 to 35 nucleotides in length in positions −578 to −386 (SEQ ID NO: 2), positions −339 to −291 (SEQ ID NO: 3), positions −162 to −86 (SEQ ID NO: 4) or positions −55 to −34 (SEQ ID NO: 5) upstream of the transcription start site in a promoter region of an ATOH1 gene, wherein the first nucleic acid strand and the second nucleic acid strand complementarily form a double-stranded nucleic acid structure.

2. The small activating nucleic acid molecule of claim 1, wherein the first nucleic acid strand and the second nucleic acid strand are present on two different nucleic acid strands.

3. The small activating nucleic acid molecule of claim 1, wherein the first nucleic acid strand and the second nucleic acid strand are present on the same nucleic acid strand, and the small activating nucleic acid molecule is a hairpin single-stranded nucleic acid molecule, wherein the first nucleic acid strand and the second nucleic acid strand comprise complementary regions forming the double-stranded nucleic acid structure.

4. (canceled)

5. The small activating nucleic acid molecule of claim 2, wherein both strands of the small activating nucleic acid molecule have overhangs of of 2 or 3 nucleotides in length at the 3′ terminus.

6. The small activating nucleic acid molecule of claim 1, wherein the first nucleic acid strand and the second nucleic acid strand independently have 16 to 35 nucleotides in length.

7. The small activating nucleic acid molecule of claim 1, wherein one strand of the small activating nucleic acid molecule comprises a nucleic acid sequence having at least 75% homology or complementarity to a nucleotide sequence selected from the group consisting of SEQ ID NOs: 280-416, or consists of a nucleic acid sequence having at least 75% homology or complementarity to a nucleotide sequence selected from the group consisting of SEQ ID NOs: 280-416.

8. The small activating nucleic acid molecule of claim 1, wherein the first nucleic acid strand has at least 75% homology to a nucleotide sequence selected from the group consisting of SEQ ID NOs: 6-142, and the second nucleic acid strand has at least 75% homology to a nucleotide sequence selected from the group consisting of SEQ ID NOs: 143-279.

9. The small activating nucleic acid molecule of claim 1, wherein said first and second strand is selected from the group consisting of:
SEQ ID NO: 6 and SEQ ID NO: 143;
SEQ ID NO: 7 and SEQ ID NO: 144;
SEQ ID NO: 8 and SEQ ID NO: 145;
SEQ ID NO: 9 and SEQ ID NO: 146;
SEQ ID NO: 10 and SEQ ID NO: 147;
SEQ ID NO: 11 and SEQ ID NO: 148;
SEQ ID NO: 12 and SEQ ID NO: 149;
SEQ ID NO: 13 and SEQ ID NO: 150;
SEQ ID NO: 14 and SEQ ID NO: 151;
SEQ ID NO: 15 and SEQ ID NO: 152;
SEQ ID NO: 16 and SEQ ID NO: 153;
SEQ ID NO: 17 and SEQ ID NO: 154;
SEQ ID NO: 18 and SEQ ID NO: 155;
SEQ ID NO: 19 and SEQ ID NO: 156;
SEQ ID NO: 20 and SEQ ID NO: 157;
SEQ ID NO: 21 and SEQ ID NO: 158;
SEQ ID NO: 22 and SEQ ID NO: 159;
SEQ ID NO: 23 and SEQ ID NO: 160;
SEQ ID NO: 24 and SEQ ID NO: 161;
SEQ ID NO: 25 and SEQ ID NO: 162;
SEQ ID NO: 26 and SEQ ID NO: 163;
SEQ ID NO: 27 and SEQ ID NO: 164;
SEQ ID NO: 28 and SEQ ID NO: 165;
SEQ ID NO: 29 and SEQ ID NO: 166;
SEQ ID NO: 30 and SEQ ID NO: 167;
SEQ ID NO: 31 and SEQ ID NO: 168;
SEQ ID NO: 32 and SEQ ID NO: 169;
SEQ ID NO: 33 and SEQ ID NO: 170;
SEQ ID NO: 34 and SEQ ID NO: 171;
SEQ ID NO: 35 and SEQ ID NO: 172;
SEQ ID NO: 36 and SEQ ID NO: 173;
SEQ ID NO: 37 and SEQ ID NO: 174;
SEQ ID NO: 38 and SEQ ID NO: 175;
SEQ ID NO: 39 and SEQ ID NO: 176;
SEQ ID NO: 40 and SEQ ID NO: 177;
SEQ ID NO: 41 and SEQ ID NO: 178;
SEQ ID NO: 42 and SEQ ID NO: 179;
SEQ ID NO: 43 and SEQ ID NO: 180;
SEQ ID NO: 44 and SEQ ID NO: 181;
SEQ ID NO: 45 and SEQ ID NO: 182;
SEQ ID NO: 46 and SEQ ID NO: 183;
SEQ ID NO: 47 and SEQ ID NO: 184;
SEQ ID NO: 48 and SEQ ID NO: 185;
SEQ ID NO: 49 and SEQ ID NO: 186;
SEQ ID NO: 50 and SEQ ID NO: 187;
SEQ ID NO: 51 and SEQ ID NO: 188;
SEQ ID NO: 52 and SEQ ID NO: 189;
SEQ ID NO: 53 and SEQ ID NO: 190;
SEQ ID NO: 54 and SEQ ID NO: 191;
SEQ ID NO: 55 and SEQ ID NO: 192;
SEQ ID NO: 56 and SEQ ID NO: 193;
SEQ ID NO: 57 and SEQ ID NO: 194;
SEQ ID NO: 58 and SEQ ID NO: 195;
SEQ ID NO: 59 and SEQ ID NO: 196;
SEQ ID NO: 60 and SEQ ID NO: 197;
SEQ ID NO: 61 and SEQ ID NO: 198;
SEQ ID NO: 62 and SEQ ID NO: 199;
SEQ ID NO: 63 and SEQ ID NO: 200;
SEQ ID NO: 64 and SEQ ID NO: 201;
SEQ ID NO: 65 and SEQ ID NO: 202;
SEQ ID NO: 66 and SEQ ID NO: 203;
SEQ ID NO: 67 and SEQ ID NO: 204;
SEQ ID NO: 68 and SEQ ID NO: 205;
SEQ ID NO: 69 and SEQ ID NO: 206;
SEQ ID NO: 70 and SEQ ID NO: 207;
SEQ ID NO: 71 and SEQ ID NO: 208;
SEQ ID NO: 72 and SEQ ID NO: 209;
SEQ ID NO: 73 and SEQ ID NO: 210;
SEQ ID NO: 74 and SEQ ID NO: 211;
SEQ ID NO: 75 and SEQ ID NO: 212;
SEQ ID NO: 76 and SEQ ID NO: 213;
SEQ ID NO: 77 and SEQ ID NO: 214;
SEQ ID NO: 78 and SEQ ID NO: 215;
SEQ ID NO: 79 and SEQ ID NO: 216;
SEQ ID NO: 80 and SEQ ID NO: 217;
SEQ ID NO: 81 and SEQ ID NO: 218;
SEQ ID NO: 82 and SEQ ID NO: 219;
SEQ ID NO: 83 and SEQ ID NO: 220;
SEQ ID NO: 84 and SEQ ID NO: 221;
SEQ ID NO: 85 and SEQ ID NO: 222;
SEQ ID NO: 86 and SEQ ID NO: 223;
SEQ ID NO: 87 and SEQ ID NO: 224;
SEQ ID NO: 88 and SEQ ID NO: 225;
SEQ ID NO: 89 and SEQ ID NO: 226;
SEQ ID NO: 90 and SEQ ID NO: 227;
SEQ ID NO: 91 and SEQ ID NO: 228;
SEQ ID NO: 92 and SEQ ID NO: 229;
SEQ ID NO: 93 and SEQ ID NO: 230;
SEQ ID NO: 94 and SEQ ID NO: 231;
SEQ ID NO: 95 and SEQ ID NO: 232;
SEQ ID NO: 96 and SEQ ID NO: 233;
SEQ ID NO: 97 and SEQ ID NO: 234;
SEQ ID NO: 98 and SEQ ID NO: 235;
SEQ ID NO: 99 and SEQ ID NO: 236;
SEQ ID NO: 100 and SEQ ID NO: 237;
SEQ ID NO: 101 and SEQ ID NO: 238;
SEQ ID NO: 102 and SEQ ID NO: 239;
SEQ ID NO: 103 and SEQ ID NO: 240;
SEQ ID NO: 104 and SEQ ID NO: 241;
SEQ ID NO: 105 and SEQ ID NO: 242;
SEQ ID NO: 106 and SEQ ID NO: 243;
SEQ ID NO: 107 and SEQ ID NO: 244;
SEQ ID NO: 108 and SEQ ID NO: 245;
SEQ ID NO: 109 and SEQ ID NO: 246;
SEQ ID NO: 110 and SEQ ID NO: 247;
SEQ ID NO: 111 and SEQ ID NO: 248;
SEQ ID NO: 112 and SEQ ID NO: 249;
SEQ ID NO: 113 and SEQ ID NO: 250;
SEQ ID NO: 114 and SEQ ID NO: 251;
SEQ ID NO: 115 and SEQ ID NO: 252;
SEQ ID NO: 116 and SEQ ID NO: 253;
SEQ ID NO: 117 and SEQ ID NO: 254;
SEQ ID NO: 118 and SEQ ID NO: 255;
SEQ ID NO: 119 and SEQ ID NO: 256;
SEQ ID NO: 120 and SEQ ID NO: 257;
SEQ ID NO: 121 and SEQ ID NO: 258;
SEQ ID NO: 122 and SEQ ID NO: 259;
SEQ ID NO: 123 and SEQ ID NO: 260;
SEQ ID NO: 124 and SEQ ID NO: 261;
SEQ ID NO: 125 and SEQ ID NO: 262;
SEQ ID NO: 126 and SEQ ID NO: 263;
SEQ ID NO: 127 and SEQ ID NO: 264;
SEQ ID NO: 128 and SEQ ID NO: 265;
SEQ ID NO: 129 and SEQ ID NO: 266;
SEQ ID NO: 130 and SEQ ID NO: 267;
SEQ ID NO: 131 and SEQ ID NO: 268;
SEQ ID NO: 132 and SEQ ID NO: 269;
SEQ ID NO: 133 and SEQ ID NO: 270;
SEQ ID NO: 134 and SEQ ID NO: 271;
SEQ ID NO: 135 and SEQ ID NO: 272;
SEQ ID NO: 136 and SEQ ID NO: 273;
SEQ ID NO: 137 and SEQ ID NO: 274;
SEQ ID NO: 138 and SEQ ID NO: 275;
SEQ ID NO: 139 and SEQ ID NO: 276;
SEQ ID NO: 140 and SEQ ID NO: 277;
SEQ ID NO: 141 and SEQ ID NO: 278; and
SEQ ID NO: 142 and SEQ ID NO: 279.

10. (canceled)

11. The small activating nucleic acid molecule of claim 1, wherein the small activating nucleic acid molecule comprises at least one chemical modification selected from the group consisting of:
(1) a modification of a phosphodiester bond connecting nucleotides in the nucleotide sequence of the small activating nucleic acid molecule;
(2) a modification of 2′-OH of a ribose in the nucleotide sequence of the small activating nucleic acid molecule; (3) a modification of a base in the nucleotide sequence of the small activating nucleic acid molecule; and
(4) at least one nucleotide in the nucleotide sequence of the small activating nucleic acid molecule being a locked nucleic acid.

12. The small activating nucleic acid molecule of claim 11, wherein the at least one chemical modification is one or more modifications selected from the group consisting of: 2′-fluoro modification, 2′-oxymethyl modification, 2′-oxyethylidene methoxy modification, 2,4′-dinitrophenol modification, locked nucleic acid (LNA), 2′-amino modification, 2′-deoxy modification, 5′-bromouracil modification, 5′-iodouracil modification, N-methyluracil modification, 2,6-diaminopurine modification, phosphorothioate modification, and boranophosphate modification.

13. The small activating nucleic acid molecule of claim 1, wherein the small activating nucleic acid molecule activates or upregulates the expression of the ATOH 1 gene by at least 10%.

14. A nucleic acid encoding the small activating nucleic acid molecule of claim 1.

15. (canceled)

16. The small activating nucleic acid molecule of claim 1, wherein the first nucleic acid strand has at least 95% homology to a nucleotide sequence selected from the group consisting of SEQ ID NOs: 6-142, and the second nucleic acid strand has at least 95% homology to a nucleotide sequence selected from the group consisting of SEQ ID NOs:
143-279.

17. A composition comprising the small activating nucleic acid molecule of claim 1, and a pharmaceutically acceptable carrier.

18. (canceled)

19. (canceled)

20. The small activating nucleic acid molecule of claim 8, wherein all the nucleotides are natural non-chemically modified nucleotides.

21-29. (canceled)

30. A method for activating or upregulating the expression of an ATOH1 gene in a cell, comprising administering the small activating nucleic acid molecule of claim 1 to the cell.

31-34. (canceled)

35. The method of claim 30, wherein the cell is present in a human body.

36. (canceled)

37. (canceled)

38. A method for treating a disease or condition related to insufficient or decreased expression of the ATOH1 protein in an individual, comprising administering the small activating nucleic acid molecule of claim 1 to the individual.

39. The method of claim 38, wherein the disease or condition related to insufficient or decreased expression of the ATOH1 protein comprises hearing loss and tumor.

40. (canceled)

41. The method of claim 38, wherein the individual is a human.

42-46. (canceled)
